(19) United States
(12) Patent Application Publication
(10) Pub. No.: US 2013/0165490 A1
(43) Pub. Date: Jun. 27, 2013

(54) INSECTICIDAL COMPOUNDS

(71) Applicants: Myriem El Qacemi, Stein (CH); Thomas Pitterna, Stein (CH); Jerome Yves Cassayre, Stein (CH); Peter Renold, Stein (CH)

(72) Inventors: Myriem El Qacemi, Stein (CH); Thomas Pitterna, Stein (CH); Jerome Yves Cassayre, Stein (CH); Peter Renold, Stein (CH)

(73) Assignees: Myriem El Qacemi, Stein (CH); Thomas Pitterna, Stein (CH); Syngenta Crop Protection LLC, Greensboro, NC (US); Peter Renold, Stein (CH); Jerome Yves Cassayre, Stein (CH)

(21) Appl. No.: 13/772,961

(22) Filed: Feb. 21, 2013

Related U.S. Application Data
(63) Continuation of application No. 13/147,054, filed on Jul. 29, 2011, now abandoned.

(30) Foreign Application Priority Data
Jan. 29, 2009 (GB) 0901508.2
Jun. 22, 2009 (GB) 0910767.3
Jun. 22, 2009 (GB) 0910769.9
Jul. 24, 2009 (EP) PCT/EP2009/059563

(51) Int. Cl. C07D 261/06; C07D 261/14; C07D 413/12; A01N 43/80

(52) U.S. Cl. CPC C07D 261/06; A01N 43/80; C07D 261/14; C07D 413/12
USPC 514/378; 514/380; 548/245; 548/247

(57) ABSTRACT

A compound of formula (I):

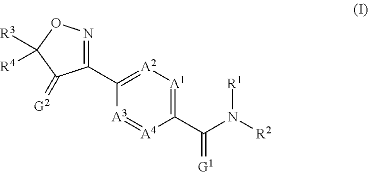

wherein A¹, A², A³, A⁴, G¹, G², R¹, R², R³ and R⁴ are as defined in claim 1; or a salt or N-oxide thereof.

Furthermore, the present invention relates to processes and intermediates for preparing compounds of formula (I), to insecticidal, acaricidal, nematicidal and molluscicidal compositions comprising them and to methods of using them to combat and control insect, acarine, nematode and mollusc pests.

The present invention relates to certain benzamide isoxazolines, to processes and intermediates for preparing them, to insecticidal, acaricidal, nematicidal and molluscicidal compositions comprising them and to methods of using them to combat and control insect, acarine, nematode and mollusc pests.

Certain isoxazoline derivatives with insecticidal properties are disclosed, for example, in WO 2009/005015.

It has now surprisingly been found that certain benzamide isoxazolines with a substituent in the 4-position of the isoxazoline ring have insecticidal properties.

The present invention therefore provides a compound of formula (I)

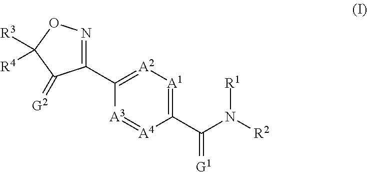

wherein
A¹, A², A³ and A⁴ are independently of each other C—H, C—R⁵, or nitrogen;
G¹ is oxygen or sulfur;
G² is C(R^{6a})(R^{6b}), oxygen, sulfur, or N—R⁷;
R¹ is hydrogen, C₁-C₈alkyl, C₁-C₈alkoxy-, C₁-C₈alkylcarbonyl-, or C₁-C₈alkoxycarbonyl-;
R² is C₁-C₈alkyl or C₁-C₈alkyl substituted by one to five R⁸, C₃-C₁₀cycloalkyl or C₃-C₁₀cycloalkyl substituted by one to five R⁹, aryl-C₁-C₄alkylene- or aryl-C₁-C₄alkylene- wherein the aryl moiety is substituted by one to five R¹⁰, heterocyclyl-C₁-C₄alkylene- or heterocyclyl-C₁-C₄alkylene- wherein the heterocyclyl moiety is substituted by one to five R¹⁰, aryl or aryl substituted by one to five R¹⁰, heterocyclyl or heterocyclyl substituted by one to five R¹⁰, C₁-C₈alkylaminocarbonyl-C₁-C₄ alkylene, C₁-C₈haloalkylaminocarbonyl-C₁-C₄ alkylene, or C₃-C₈cycloalkyl-aminocarbonyl-C₁-C₄ alkylene;
R³ is C₁-C₈haloalkyl;
R⁴ is aryl or aryl substituted by one to five R¹¹, or heteroaryl or heteroaryl substituted by one to five R¹¹;
each R⁵ is independently halogen, cyano, nitro, C₁-C₈alkyl, C₁-C₈haloalkyl, C₃-C₁₀cycloalkyl, C₁-C₈alkoxy-, C₁-C₈haloalkoxy-, C₁-C₈alkylthio-, C₁-C₈haloalkylthio-, C₁-C₈alkylsulfinyl-, C₁-C₈haloalkylsulfinyl-, C₁-C₈alkylsulfonyl-, or C₁-C₈haloalkylsulfonyl-;
R^{6a} and R^{6b} are independently of each other hydrogen, halogen, C₁-C₈alkyl or C₁-C₈alkyl substituted by one to five R¹², C₂-C₈alkenyl or C₂-C₈alkenyl substituted by one to five R¹², C₂-C₈ alkynyl, C₂-C₈haloalkynyl, C₁-C₈ alkoxy-, C₁-C₈haloalkoxy-, C₁-C₈ alkylcarbonyl-, C₁-C₈alkoxycarbonyl-, aryl or aryl substituted by one to five R¹³, or heterocyclyl or heterocyclyl substituted by one to five R¹³, or NR¹⁴R¹⁵, wherein
R¹⁴ and R¹⁵ are independently hydrogen, C₁-C₈ alkyl, C₁-C₈haloalkyl, C₁-C₈ alkylcarbonyl-, C₁-C₈ alkoxycarbonyl-, or
R¹⁴ and R¹⁵ together with the nitrogen atom to which they are attached form a 3 to 7 membered heterocyclic ring; or
R^{6a} and R^{6b} together with the carbon atom to which they are attached form a 3 to 7 membered carbocyclic or heterocyclic ring;
R⁷ is hydrogen, hydroxy, C₁-C₈alkyl or C₁-C₈alkyl substituted by one to five R¹⁶, C₃-C₁₀cycloalkyl, C₁-C₈ alkoxy- or C₁-C₈ alkoxy-substituted by one to five R¹⁶, (C₁-C₈ alkyl)amino-, di(C₁-C₈alkyl)amino-, (C₁-C₈ alkylcarbonyl)amino-, or (C₁-C₈ alkoxycarbonyl)amino-;
each R⁸, R¹² and R¹⁶ is independently halogen, cyano, nitro, hydroxy, C₁-C₈alkoxy-, C₁-C₈haloalkoxy-, C₁-C₈ alkylcarbonyl-, C₁-C₈ alkoxycarbonyl-, mercapto, C₁-C₈ alkylthio-, C₁-C₈haloalkylthio-, C₁-C₈ alkylsulfinyl-, C₁-C₈haloalkylsulfinyl-, C₁-C₈ alkylsulfonyl-, C₁-C₈haloalkylsulfonyl-;
each R⁹ is independently halogen or C₁-C₈alkyl;
each R¹⁰, R¹¹ and R¹³ is independently halogen, cyano, nitro, C₁-C₈alkyl, C₁-C₈haloalkyl, C₂-C₈ alkenyl, C₂-C₈haloalkenyl, C₂-C₈ alkynyl, C₂-C₈haloalkynyl, hydroxy, C₁-C₈ alkoxy-, C₁-C₈haloalkoxy-, mercapto, C₁-C₈ alkylthio-, C₁-C₈haloalkylthio-, C₁-C₈ alkylsulfinyl-, C₁-C₈haloalkylsulfinyl-, C₁-C₈ alkylsulfonyl-, C₁-C₈haloalkylsulfonyl-, C₁-C₈ alkylcarbonyl-, C₁-C₈alkoxycarbonyl-, aryl or aryl substituted by one to five R¹⁷, or heterocyclyl or heterocyclyl substituted by one to five R¹⁷;
each R¹⁷ is independently halogen, cyano, nitro, C₁-C₄alkyl, C₁-C₄haloalkyl, C₁-C₄alkoxy-, or C₁-C₄haloalkoxy-; or a salt or N-oxide thereof.

The compounds of formula (I) may exist in different geometric or optical isomers or tautomeric forms. This invention covers all such isomers and tautomers and mixtures thereof in all proportions as well as isotopic forms such as deuterated compounds.

The compounds of the invention may contain one or more asymmetric carbon atoms, for example, at the —CR³R⁴— group, and may exist as enantiomers (or as pairs of diastereo-isomers) or as mixtures of such.

Alkyl groups (either alone or as part of a larger group, such as alkoxy-, alkylthio-, alkylsulfinyl-, alkylsulfonyl-, alkylcarbonyl-, alkoxycarbonyl- or alkylamino-) can be in the form of a straight or branched chain and are, for example, methyl, ethyl, propyl, prop-2-yl, butyl, but-2-yl, 2-methyl-prop-1-yl or 2-methyl-prop-2-yl. The alkyl groups are preferably C₁-C₆, more preferably C₁-C₄, most preferably C₁-C₃ alkyl groups. Where an alkyl moiety is said to be substituted, the alkyl moiety is preferably substituted by one to four substituents, most preferably by one to three substituents.

Alkylene groups can be in the form of a straight or branched chain and are, for example, —CH₂—, —CH₂—CH₂—, —CH(CH₃)—, —CH₂—CH₂—CH₂—, —CH(CH₃)—CH₂—, or —CH(CH₂CH₃)—. The alkylene groups are preferably C₁-C₃, more preferably C₁-C₂, most preferably C₁ alkylene groups.

Alkenyl groups can be in the form of straight or branched chains, and can be, where appropriate, of either the (E)- or (Z)-configuration. Examples are vinyl and allyl. The alkenyl groups are preferably C₂-C₆, more preferably C₂-C₄, most preferably C₂-C₃ alkenyl groups. Where an alkenyl moiety is said to be substituted, the alkenyl moiety is preferably substituted by one to four substituents, most preferably by one to three substituents.

Alkynyl groups can be in the form of straight or branched chains. Examples are ethynyl and propargyl. The alkynyl groups are preferably C₂-C₆, more preferably C₂-C₄, most preferably C₂-C₃ alkynyl groups.

Halogen is fluorine, chlorine, bromine or iodine.

Haloalkyl groups (either alone or as part of a larger group, such as haloalkoxy-, haloalkylthio-, haloalkylsulfinyl- or haloalkylsulfonyl-) are alkyl groups which are substituted by one or more of the same or different halogen atoms and are, for example, difluoromethyl, trifluoromethyl, chlorodifluoromethyl or 2,2,2-trifluoro-ethyl.

Haloalkenyl groups are alkenyl groups which are substituted by one or more of the same or different halogen atoms and are, for example, 2,2-difluoro-vinyl or 1,2-dichloro-2-fluoro-vinyl.

Haloalkynyl groups are alkynyl groups which are substituted by one or more of the same or different halogen atoms and are, for example, 1-chloro-prop-2-ynyl.

Cycloalkyl groups or carbocyclic rings can be in mono- or bi-cyclic form and are, for example, cyclopropyl, cyclobutyl, cyclohexyl and bicyclo[2.2.1]heptan-2-yl. The cycloalkyl groups are preferably C₃-C₈, more preferably C₃-C₆ cycloalkyl groups. Where a cycloalkyl moiety is said to be substituted, the cycloalkyl moiety is preferably substituted by one to four substituents, most preferably by one to three substituents.

Aryl groups (either alone or as part of a larger group, such as aryl-alkylene-) are aromatic ring systems which can be in mono-, bi- or tricyclic form. Examples of such rings include phenyl, naphthyl, anthracenyl, indenyl or phenanthrenyl. Preferred aryl groups are phenyl and naphthyl, phenyl being most preferred. Where an aryl moiety is said to be substituted, the aryl moiety is preferably substituted by one to four substituents, most preferably by one to three substituents.

Heteroaryl groups (either alone or as part of a larger group, such as heteroaryl-alkylene-) are aromatic ring system containing at least one heteroatom and consisting either of a single ring or of two or more fused rings. Preferably, single rings will contain up to three heteroatoms and bicyclic systems up to four heteroatoms which will preferably be chosen from nitrogen, oxygen and sulfur. Examples of monocyclic groups include pyridyl, pyridazinyl, pyrimidinyl, pyrazinyl, pyrrolyl, pyrazolyl, imidazolyl, triazolyl, furanyl, thiophenyl, oxazolyl, isoxazolyl, oxadiazolyl, thiazolyl, isothiazolyl and thiadiazolyl. Examples of bicyclic groups include quinolinyl, cinnolinyl, quinoxalinyl, indolyl, indazolyl, benzimidazolyl, benzothiophenyl and benzothiazolyl. Monocyclic heteroaryl groups are preferred, pyridyl being most preferred. Where a heteroaryl moiety is said to be substituted, the heteroaryl moiety is preferably substituted by one to four substituents, most preferably by one to three substituents.

Heterocyclyl groups or heterocyclic rings (either alone or as part of a larger group, such as heterocyclyl-alkylene-) are defined to include heteroaryl groups and in addition their unsaturated or partially unsaturated analogues. Examples of monocyclic groups include thietanyl, pyrrolidinyl, tetrahydrofuranyl, [1,3]dioxolanyl, piperidinyl, piperazinyl, [1,4]dioxanyl, and morpholinyl or their oxidised versions such as 1-oxo-thietanyl and 1,1-dioxo-thietanyl. Examples of bicyclic groups include 2,3-dihydro-benzofuranyl, benzo[1,3]dioxolanyl, and 2,3-dihydro-benzo[1,4]dioxinyl. Where a heterocyclyl moiety is said to be substituted, the heterocyclyl moiety is preferably substituted by one to four substituents, most preferably by one to three substituents.

Preferred values of A¹, A², A³, A⁴, G¹, G², R¹, R², R³, R⁴, R⁵, R^{6a}, R^{6b}, R⁷, R⁸, R⁹, R¹⁰, R¹¹, R¹², R¹³, R¹⁴, R¹⁵, R¹⁶ and R¹⁷ are, in any combination, as set out below.

Preferably no more than two of A¹, A², A³ and A⁴ are nitrogen.

Preferably A¹ is C—H or C—R⁵, most preferably A¹ is C—R⁵.

Preferably A² is C—H or C—R⁵, most preferably A² is C—H.

Preferably A³ is C—H or C—R⁵, most preferably A³ is C—H.

Preferably A⁴ is C—H or C—R⁵, most preferably A⁴ is C—H.

Preferably A¹ is C—R⁵, A² is CH, A³ is CH or nitrogen and A⁴ is CH or nitrogen.

Preferably A¹ is C—R⁵, A² is CH, A³ is CH and A⁴ is CH.

Preferably G¹ is oxygen.

Preferably G² is C(R^{6a})(R^{6b}), oxygen or N—R⁷, more preferably C(H)(R^{6b}), oxygen or N—R⁷, more preferably C(H)(C₁-C₆alkyl), C(H)(phenyl), oxygen, N(OH), N(OC₁-C₆alkyl), or N(OC₁-C₆haloalkyl), more preferably C(H)(C₁-C₆alkyl), oxygen, N(OH) or N(OC₁-C₆alkyl), more preferably C(H)(C₁-C₆alkyl), oxygen, or N(OH), more preferably C(H)(CH₃), C(H)(phenyl), oxygen, or N(OH), most preferably C(H)(CH₃).

Preferably R¹ is hydrogen, methyl, ethyl, methylcarbonyl-, or methoxycarbonyl-, more preferably hydrogen, methyl or ethyl, even more preferably hydrogen or methyl, most preferably hydrogen.

Preferably R² is C₁-C₈alkyl or C₁-C₈alkyl substituted by one to five R⁸, C₃-C₁₀cycloalkyl or C₃-C₁₀cycloalkyl substituted by one to five R⁹, aryl-C₁-C₄alkylene- or aryl-C₁-C₄alkylene- wherein the aryl moiety is substituted by one to five R¹⁰, heterocyclyl-C₁-C₄alkylene- or heterocyclyl-C₁-C₄alkylene- wherein the heterocyclyl moiety is substituted by one to five R¹⁰, aryl or aryl substituted by one to five R¹⁰, or heterocyclyl or heterocyclyl substituted by one to five R¹⁰, C₁-C₈alkylaminocarbonyl-C₁-C₄ alkylene, C₁-C₈haloalkylaminocarbonyl-C₁-C₄ alkylene, or C₃-C₈cycloalkyl-aminocarbonyl-C₁-C₄ alkylene, wherein each aryl group is a phenyl group and each heterocycle group is selected from pyridyl, pyridazinyl, pyrimidinyl, pyrazinyl, pyrrolyl, pyrazolyl, imidazolyl, triazolyl, furanyl, thiophenyl, oxazolyl, isoxazolyl, oxadiazolyl, thiazolyl, isothiazolyl, thiadiazolyl, quinolinyl, cinnolinyl, quinoxalinyl, indolyl, indazolyl, benzimidazolyl, benzothiophenyl, benzothiazolyl, oxetanyl, thietanyl, oxo-thietanyl, dioxo-thietanyl, pyrrolidinyl, tetrahydrofuranyl, [1,3]dioxolanyl, piperidinyl, piperazinyl, [1,4]dioxanyl, and morpholinyl, 2,3-dihydro-benzofuranyl, benzo[1,3]dioxolanyl, and 2,3-dihydro-benzo[1,4]dioxinyl.

Preferably R² is C₁-C₈alkyl or C₁-C₈alkyl substituted by one to five R⁸, C₃-C₁₀cycloalkyl or C₃-C₁₀cycloalkyl substituted by one to five R⁹, aryl-C₁-C₄alkylene- or aryl-C₁-C₄alkylene- wherein the aryl moiety is substituted by one to five R¹⁰, heterocyclyl-C₁-C₄alkylene- or heterocyclyl-C₁-C₄alkylene- wherein the heterocyclyl moiety is substituted by one to five R¹⁰, aryl or aryl substituted by one to five R¹⁰, heterocyclyl or heterocyclyl substituted by one to five R¹⁰, C₁-C₈alkylaminocarbonyl-C₁-C₄ alkylene, C₁-C₈haloalkylaminocarbonyl-C₁-C₄ alkylene, or C₃-C₈cycloalkyl-aminocarbonyl-C₁-C₄ alkylene, wherein each aryl group is a phenyl group and each heterocycle group is selected from pyridyl, pyrazolyl, benzimidazolyl, furanyl, thiazolyl, oxetanyl, thietanyl, oxo-thietanyl and dioxo-thietanyl.

Preferably R² is C₁-C₈alkyl or C₁-C₈alkyl substituted by one to five R⁸, C₃-C₁₀cyclo-alkyl or C₃-C₁₀cycloalkyl substituted by one to five R⁹, phenyl-C₁-C₄alkylene- or phenyl-C₁-C₄alkylene- wherein the phenyl moiety is substituted by one to five R¹⁰, pyridyl-C₁-C₄alkylene- or pyridyl-C₁-C₄alkylene- wherein the pyridyl moiety is substituted by one to four R¹⁰, oxetanyl or oxetanyl substituted by one to five R¹⁰, thietanyl or thietanyl substituted by one to five R¹⁰, oxo-thietanyl or oxo-thietanyl substituted by one to five R¹⁰, dioxo-thietanyl or dioxo-thietanyl substituted by one to five R¹⁰, C₁-C₈alkylaminocarbonyl-C₁-C₄ alkylene, C₁-C₈haloalkylaminocarbonyl-C₁-C₄ alkylene, or C₃-C₈cycloalkyl-aminocarbonyl-C₁-C₄ alkylene, for example C₁-C₈alkyl or C₁-C₈alkyl substituted by one to five R⁸, C₃-C₁₀cycloalkyl or C₃-C₁₀cycloalkyl substituted by one to five R⁹, phenyl-C₁-C₄alkylene- or phenyl-C₁-C₄alkylene- wherein the phenyl moiety is substituted by one to five R¹⁰, pyridyl-C₁-C₄alkylene- or pyridyl-C₁-C₄alkylene- wherein the pyridyl moiety is substituted by one to four R¹⁰, oxetanyl or oxetanyl substituted by one to five R¹⁰, thietanyl or thietanyl substituted by one to five R¹⁰, oxo-thietanyl or oxo-thietanyl substituted by one to five R¹⁰, dioxo-thietanyl or dioxo-thietanyl substituted by one to five R¹⁰, more preferably C₁-C₈alkyl or C₁-C₈alkyl substituted by halogen, C₃-C₁₀cycloalkyl or C₃-C₁₀cycloalkyl substituted by one or two methyl groups, phenyl-C₁-C₄alkylene- or phenyl-C₁-C₄alkylene- wherein the phenyl moiety is substituted by one to five R¹⁰, pyridyl-C₁-C₄alkylene- or pyridyl-C₁-C₄alkylene- wherein the pyridyl moiety is substituted by one to four R¹⁰, oxetanyl, thietanyl, oxo-thietanyl, dioxo-thietanyl, C₁-C₈alkylaminocarbonyl-methylene, C₁-C₈haloalkylaminocarbonyl-methylene, or C₃-C₈cycloalkyl-aminocarbonyl-methylene, for example C₁-C₈alkyl or C₁-C₈alkyl substituted by halogen, C₃-C₁₀cycloalkyl or C₃-C₁₀cycloalkyl substituted by one or two methyl groups, phenyl-C₁-C₄alkylene- or phenyl-C₁-C₄alkylene- wherein the phenyl moiety is substituted by one to five R¹⁰, pyridyl-C₁-C₄alkylene- or pyridyl-C₁-C₄alkylene- wherein the pyridyl moiety is substituted by one to four R¹⁰, oxetanyl, thietanyl, oxo-thietanyl, dioxo-thietanyl, most preferably butyl-, cyclobutyl-, 1-phenyl-eth-1-yl-, phenyl-methyl-, (pyrid-2-yl)-methyl-, thietanyl-, -(2,2,2-trifluoro-ethyl)-acetamide-2-yl, oxo-thietanyl- or dioxo-thietanyl-, for example. butyl-, cyclobutyl-, 1-phenyl-eth-1-yl-, phenyl-methyl-, (pyrid-2-yl)-methyl-, thietanyl-, oxo-thietanyl- or dioxo-thietanyl-.

A group of preferred compounds are those wherein R² is C₁-C₆alkyl or C₁-C₆alkyl substituted by one to five R⁸, for example ethyl-, butyl-, but-2-yl-, 3-bromo-propyl-, 2,2,2-trifluoro-ethyl-, 3,3,3-trifluoro-propyl-, 2-methoxy-ethyl-, -(2,2,2-trifluoro-ethyl)-acetamide-2-yl and 1-methoxy-prop-2-yl-, for example ethyl-, butyl-, but-2-yl-, 3-bromo-propyl-, 2,2,2-trifluoro-ethyl-, 3,3,3-trifluoro-propyl-, 2-methoxy-ethyl-, and 1-methoxy-prop-2-yl-.

A group of preferred compounds are those wherein R² is C₃-C₈cycloalkyl or C₃-C₈cycloalkyl substituted by one to five R⁹, for example cyclobutyl-, and 2-methyl-cyclohex-1-yl-.

A group of preferred compounds are those wherein R² is aryl-C₁-C₂alkylene- or aryl-C₁-C₂alkylene- wherein the aryl moiety is substituted by one to five R¹⁰, for example phenyl-methyl-, 1-phenyl-eth-1-yl-, 2-phenyl-eth-1-yl-, (3-chloro-phenyl)-methyl-, (2-fluoro-phenyl)-methyl-, (4-methoxy-phenyl)-methyl-, (2-trifluoromethyl-phenyl)-methyl-, and (2-trifluoromethoxy-phenyl)-methyl-.

A group of preferred compounds are those wherein R² is heterocyclyl-C₁-C₂alkylene- or heterocyclyl-C₁-C₂alkylene- wherein the heterocyclyl moiety is substituted by one to five R¹⁰, for example (pyrid-2-yl)-methyl-, (pyrid-3-yl)-methyl-, (2-chloro-pyrid-5-yl)-methyl (1-methyl-1H-imidazol-4-yl)-methyl-, (furan-2-yl)-methyl-, 2-(thiophen-2′-yl)-eth-1-yl-, 2-(indol-3′-yl)-eth-1-yl-, (1H-benzimidazol-2-yl)-methyl-, (oxetan-2-yl)-methyl-, (tetrahydro-furan-2-yl)-methyl-, 2-([1′,3′]dioxolan-2′-yl)-eth-1-yl-, 2-(morpholin-4′-yl)-eth-1-yl-, 2-(benzo[1′,3′]dioxol-5′-yl)-eth-1-yl-, and (2,3-dihydro-benzo[1,4]dioxin-6-yl)-methyl-, more preferably R² is heteroaryl-C₁-C₂alkylene- or heteroaryl-C₁-C₂alkylene- wherein the heteroaryl moiety is substituted by one to five R¹⁰.

A group of preferred compounds are those wherein R² is aryl or aryl substituted by one to five R¹⁰, for example 2-chloro-phenyl-, 3-fluoro-phenyl-, 2-methyl-phenyl-, 2-chloro-6-methyl-phenyl-, 2-trifluoromethyl-phenyl-, and 2,4-dimethoxy-phenyl-.

A group of preferred compounds are those wherein R² is heterocyclyl or heterocyclyl substituted by one to five R¹⁰, for example 3-methyl-pyrid-2-yl-, 1,3-dimethyl-1H-pyrazol-5-yl-, 4-methyl-thiazol-2-yl-, 5-methyl-thiadiazol-2-yl-, quinolin-2-yl-, quinolin-5-yl-, benzothiazol-6-yl-, 4-methyl-benzothiazol-2-yl-, thietan-3-yl-, 1-oxo-thietan-3-yl-, 1,1-dioxo-thietan-3-yl-, and 3-methyl-thietan-3-yl-, more preferably R² is oxetanyl, thietanyl, oxo-thietanyl or dioxo-thietanyl each optionally substituted by one to five R¹⁰, most preferably R² is thietanyl, oxo-thietanyl or dioxo-thietanyl each optionally substituted by one to five R¹⁰. It is particularly preferred that the oxetanyl, thietanyl, oxo-thietanyl and dioxo-thietanyl ring is linked via the 3-position.

Preferably R³ is chlorodifluoromethyl or trifluoromethyl, most preferably trifluoro-methyl.

Preferably R⁴ is aryl or aryl substituted by one to five R¹¹, more preferably aryl substituted by two to three R¹¹, more preferably phenyl substituted by two to three R¹¹, even more preferably 3,5-dibromo-phenyl-, 3,5-dichloro-phenyl-, 3,5-bis-(trifluoromethyl)-phenyl-, 3,4-dichloro-phenyl- or 3,4,5-trichloro-phenyl-, most preferably R⁴ is 3,5-dichloro-phenyl.

Preferably each R⁵ is independently halogen, cyano, nitro, C₁-C₈alkyl, C₁-C₈halo-alkyl, C₁-C₈alkoxy-, or C₁-C₈haloalkoxy-, more preferably bromo, chloro, fluoro, cyano, nitro, methyl, ethyl, trifluoromethyl, methoxy, difluoromethoxy, or trifluoromethoxy, even more preferably bromo, chloro, fluoro, nitro, or methyl, most preferably methyl.

Preferably R^{6a} is hydrogen or C₁-C₆alkyl, most preferably hydrogen.

Preferably R^{6b} is hydrogen, C₁-C₆alkyl or phenyl, more preferably C₁-C₆alkyl or phenyl, more preferably methyl or phenyl, most preferably methyl.

Preferably R⁷ is hydroxyl, C₁-C₆alkoxy- or C₁-C₆ haloalkoxy-, more preferably R⁷ is hydroxy or C₁-C₆alkoxy-, most preferably hydroxy.

Preferably each R⁸ is independently halogen, cyano, nitro, hydroxy, C₁-C₈alkoxy-, C₁-C₈haloalkoxy-, mercapto, C₁-C₈alkylthio-, C₁-C₈haloalkylthio-, more preferably bromo, chloro, fluoro, methoxy, or methylthio, most preferably chloro, fluoro, or methoxy.

Preferably each R⁹ is independently chloro, fluoro or methyl, most preferably methyl.

Preferably each R¹⁰ is independently halogen, cyano, nitro, C₁-C₈alkyl, C₁-C₈halo-alkyl, C₁-C₈alkoxy-, C₁-C₈haloalkoxy-, more preferably bromo, chloro, fluoro, cyano, nitro, methyl, ethyl, trifluoromethyl, methoxy, difluoromethoxy, or trifluoromethoxy, most preferably bromo, chloro, fluoro, cyano or methyl.

Preferably each R¹¹ is independently halogen, C₁-C₈alkoxy-, C₁-C₈haloalkoxy-, C₁-C₈alkylthio-, or C₁-C₈haloalkylthio-, more preferably bromo, chloro, fluoro, methoxy, or methylthio, most preferably bromo or chloro.

Preferably each R¹² is independently halogen, cyano, nitro, hydroxy, C₁-C₈alkoxy-, C₁-C₈haloalkoxy-, mercapto, C₁-C₈alkylthio-, C₁-C₈haloalkylthio-, more preferably bromo, chloro, fluoro, methoxy, or methylthio, most preferably chloro, fluoro, or methoxy.

Preferably each R¹³ is independently halogen, cyano, nitro, C₁-C₈alkyl, C₁-C₈halo-alkyl, C₁-C₈alkoxy-, C₁-C₈haloalkoxy-, more preferably bromo, chloro, fluoro, cyano, nitro, methyl, ethyl, trifluoromethyl, methoxy, difluoromethoxy, or trifluoromethoxy, most preferably bromo, chloro, fluoro, nitro, or methyl.

Preferably R¹⁴ and R¹⁵ are C₁-C₈alkyl or together with the nitrogen atom to which they are attached form a 4 to 6 heterocyclic ring, more preferably R¹⁴ and R¹⁵ are methyl.

Preferably each R¹⁶ is independently halogen, cyano, nitro, hydroxy, C₁-C₈alkoxy-, C₁-C₈haloalkoxy-, mercapto, C₁-C₈alkylthio-, C₁-C₈haloalkylthio-, more preferably bromo, chloro, fluoro, methoxy, or methylthio, most preferably chloro, fluoro, or methoxy.

Preferably each R¹⁷ is independently bromo, chloro, fluoro, cyano, nitro, methyl, ethyl, trifluoromethyl, methoxy, difluoromethoxy, or trifluoromethoxy, more preferably bromo, chloro, fluoro, nitro, or methyl, most preferably chloro, fluoro, or methyl.

For example, the present invention provides compounds of formula (I) wherein

G¹ is oxygen or sulfur;

G² is C(R^{6a})(R^{6b}), oxygen, sulfur, or N—R⁷;

R¹ is hydrogen, C₁-C₈alkyl, C₁-C₈alkoxy-, C₁-C₈alkylcarbonyl-, or C₁-C₈alkoxycarbonyl-;

R² is C₁-C₈alkyl or C₁-C₈alkyl substituted by one to five R⁸, C₃-C₁₀cycloalkyl or C₃-C₁₀cycloalkyl substituted by one to five R⁹, aryl-C₁-C₄alkylene- or aryl-C₁-C₄alkylene- wherein the aryl moiety is substituted by one to five R¹⁰, heterocyclyl-C₁-C₄alkylene- or heterocyclyl-C₁-C₄alkylene- wherein the heterocyclyl moiety is substituted by one to five R¹⁰, aryl or aryl substituted by one to five R¹⁰, or heterocyclyl or heterocyclyl substituted by one to five R¹⁰;

R³ is C₁-C₈haloalkyl;

R⁴ is aryl or aryl substituted by one to five R¹¹, or heteroaryl or heteroaryl substituted by one to five R¹¹;

each R⁵ is independently halogen, cyano, nitro, C₁-C₈alkyl, C₁-C₈haloalkyl, C₃-C₁₀cycloalkyl, C₁-C₈alkoxy-, C₁-C₈haloalkoxy-, C₁-C₈alkylthio-, C₁-C₈haloalkylthio-, C₁-C₈alkylsulfinyl-, C₁-C₈haloalkylsulfinyl-, C₁-C₈alkylsulfonyl-, or C₁-C₈haloalkylsulfonyl-;

R^{6a} and R^{6b} are independently of each other hydrogen, halogen, C₁-C₈alkyl or C₁-C₈alkyl substituted by one to five R¹², C₂-C₈alkenyl or C₂-C₈alkenyl substituted by one to five R¹², C₂-C₈alkynyl, C₂-C₈haloalkynyl, C₁-C₈alkoxy-, C₁-C₈haloalkoxy-, C₁-C₈alkylcarbonyl-, C₁-C₈alkoxycarbonyl-, aryl or aryl substituted by one to five R¹³, or heterocyclyl or heterocyclyl substituted by one to five R¹³, or NR¹⁴R¹⁵, wherein R¹⁴ and R¹⁵ are independently hydrogen, C₁-C₈alkyl, C₁-C₈haloalkyl, C₁-C₈alkylcarbonyl-, C₁-C₈alkoxycarbonyl-, or R¹⁴ and R¹⁵ together with the nitrogen atom to which they are attached form a 3 to 7 membered heterocyclic ring; or R^{6a} and R^{6b} together with the carbon atom to which they are attached form a 3 to 7 membered carbocyclic or heterocyclic ring;

R⁷ is hydrogen, hydroxy, C₁-C₈alkyl or C₁-C₈alkyl substituted by one to five R¹⁶, C₃-C₁₀cycloalkyl, C₁-C₈alkoxy- or C₁-C₈alkoxy- substituted by one to five R¹⁶, (C₁-C₈alkyl)amino-, di(C₁-C₈alkyl)amino-, (C₁-C₈alkylcarbonyl)amino-, or (C₁-C₈alkoxycarbonyl)amino-;

each R⁸, R¹² and R¹⁶ is independently halogen, cyano, nitro, hydroxy, C₁-C₈alkoxy-, C₁-C₈haloalkoxy-, C₁-C₈alkylcarbonyl-, C₁-C₈alkoxycarbonyl-, mercapto, C₁-C₈alkylthio-, C₁-C₈haloalkylthio-, C₁-C₈alkylsulfinyl-, C₁-C₈haloalkylsulfinyl-, C₁-C₈alkylsulfonyl-, or C₁-C₈haloalkylsulfonyl-;

each R⁹ is independently halogen or C₁-C₈alkyl;

each R¹⁰, R¹¹ and R¹³ is independently halogen, cyano, nitro, C₁-C₈alkyl, C₁-C₈haloalkyl, C₂-C₈alkenyl, C₂-C₈haloalkenyl, C₂-C₈alkynyl, C₂-C₈haloalkynyl, hydroxy, C₁-C₈alkoxy-, C₁-C₈haloalkoxy-, mercapto, C₁-C₈alkylthio-, C₁-C₈haloalkylthio-, C₁-C₈alkylsulfinyl-, C₁-C₈haloalkylsulfinyl-, C₁-C₈alkylsulfonyl-, C₁-C₈haloalkylsulfonyl-, C₁-C₈alkylcarbonyl-, C₁-C₈alkoxycarbonyl-, aryl or aryl substituted by one to five R¹⁷, or heterocyclyl or heterocyclyl substituted by one to five R¹⁷;

each R¹⁷ is independently halogen, cyano, nitro, C₁-C₄alkyl, C₁-C₄haloalkyl, C₁-C₄alkoxy-, or C₁-C₄haloalkoxy-.

For example, the present invention provides compounds of formula (I) wherein

A¹ is C—R⁵, A² is C—H, A³ is C—H or nitrogen and A⁴ is C—H or nitrogen;

G¹ is oxygen;

G² is C(R^{6a})(R^{6b}), oxygen, sulfur, or N—R⁷;

R¹ is hydrogen, methyl, ethyl, methylcarbonyl-, or methoxycarbonyl-;

R² is C₁-C₈alkyl or C₁-C₈alkyl substituted by one to five R⁸, C₃-C₁₀cycloalkyl or C₃-C₁₀cycloalkyl substituted by one to five R⁹, aryl-C₁-C₄alkylene- or aryl-C₁-C₄alkylene- wherein the aryl moiety is substituted by one to five R¹⁰, heterocyclyl-C₁-C₄alkylene- or heterocyclyl-C₁-C₄alkylene- wherein the heterocyclyl moiety is substituted by one to five R¹⁰, aryl or aryl substituted by one to five R¹⁰, heterocyclyl or heterocyclyl substituted by one to five R¹⁰, C₁-C₈alkylaminocarbonyl-C₁-C₄ alkylene, C₁-C₈haloalkylaminocarbonyl-C₁-C₄ alkylene, or C₃-C₈cycloalkyl-aminocarbonyl-C₁-C₄ alkylene, wherein each aryl group is a phenyl group and each heterocycle group is selected from pyridyl, pyrazolyl, benzimidazolyl, furanyl, thiazolyl, oxetanyl, thietanyl, oxo-thietanyl and dioxo-thietanyl.

R³ is C₁-C₈haloalkyl;

R⁴ is phenyl substituted by two to three R¹¹;

R⁵ is halogen, cyano, nitro, C₁-C₈alkyl, C₁-C₈haloalkyl, C₁-C₈alkoxy-, or C₁-C₈halo-alkoxy-;

R^{6a} is hydrogen or C₁-C₆alkyl;

R^{6b} is hydrogen, C₁-C₆alkyl or phenyl;

R⁷ is hydroxyl, C₁-C₆alkoxy- or C₁-C₆ haloalkoxy-;

each R⁸ is independently bromo, chloro, fluoro, methoxy, or methylthio;

each R⁹ is independently chloro, fluoro or methyl;

each R¹⁰ is independently halogen, cyano, nitro, C₁-C₈alkyl, C₁-C₈haloalkyl, C₁-C₈alkoxy-, C₁-C₈haloalkoxy-;

each R¹¹ is independently halogen, C₁-C₈alkoxy-, C₁-C₈haloalkoxy-, C₁-C₈alkylthio-, or C₁-C₈haloalkylthio-.

For example, the present invention provides compounds of formula (I) wherein

A¹ is C—R⁵, A² is C—H, A³ is CH and A⁴ is C—H;

G¹ is oxygen;

G² is C(R^{6a})(R^{6b}), oxygen, or N—R⁷;

R¹ is hydrogen, methyl or ethyl;

R² is C₁-C₈alkyl or C₁-C₈alkyl substituted by one to five R⁸, C₃-C₁₀cycloalkyl or C₃-C₁₀cycloalkyl substituted by one to five R⁹, phenyl-C₁-C₄alkylene- or phenyl-C₁-C₄alkylene- wherein the phenyl moiety is substituted by one to five R¹⁰, pyridyl-C₁-C₄alkylene- or pyridyl-C₁-C₄alkylene- wherein the pyridyl moiety is substituted by one to four R¹⁰, oxetanyl or oxetanyl substituted by one to five R¹⁰, thietanyl or thietanyl substituted by one to five R¹⁰, oxo-thietanyl or oxo-thietanyl substituted by one to five R¹⁰, dioxo-thietanyl or dioxo-thietanyl substituted by one to five R¹⁰, C₁-C₈alkylaminocarbonyl-C₁-C₄ alkylene, C₁-C₈haloalkylaminocarbonyl-C₁-C₄ alkylene, or C₃-C₈cycloalkyl-aminocarbonyl-C₁-C₄ alkylene;

R³ is chlorodifluoromethyl or trifluoromethyl;

R⁴ is 3,5-dibromo-phenyl-, 3,5-dichloro-phenyl-, 3,5-bis-(trifluoromethyl)-phenyl-, 3,4-dichloro-phenyl- or 3,4,5-trichloro-phenyl-;

R⁵ is bromo, chloro, fluoro, cyano, nitro, methyl, ethyl, trifluoromethyl, methoxy, difluoromethoxy, or trifluoromethoxy;

R^{6a} is hydrogen or C₁-C₆alkyl;

R^{6b} is C₁-C₆alkyl or phenyl;

R⁷ hydroxy or C₁-C₆alkoxy-;

each R⁸ is independently chloro, fluoro, or methoxy;

each R⁹ is methyl;

each R¹⁰ is independently bromo, chloro, fluoro, cyano, nitro, methyl, ethyl, trifluoromethyl, methoxy, difluoromethoxy, or trifluoromethoxy.

For example, the present invention provides compounds of formula (I) wherein

A¹ is C—R⁵, A² is C—H, A³ is C—H and A⁴ is C—H;

G¹ is oxygen;

G² is C(R^{6a})(R^{6b}), oxygen, or N—R⁷;

R¹ is hydrogen;

R² is C₁-C₈alkyl or C₁-C₈alkyl substituted by halogen, C₃-C₁₀cycloalkyl or C₃-C₁₀cycloalkyl substituted by one or two methyl groups, phenyl-C₁-C₄alkylene- or phenyl-C₁-C₄alkylene- wherein the phenyl moiety is substituted by one to five R¹⁰, pyridyl-C₁-C₄alkylene- or pyridyl-C₁-C₄alkylene- wherein the pyridyl moiety is substituted by one to four R¹⁰, oxetanyl, thietanyl, oxo-thietanyl, dioxo-thietanyl;

R³ is trifluoromethyl;

R⁴ is 3,5-dichloro-phenyl;

R⁵ is methyl;

R^{6a} is hydrogen;

R^{6b} is methyl or phenyl;

R⁷ is hydroxy;

R¹⁰ is bromo, chloro, fluoro, cyano or methyl.

For example, the present invention provides compounds of formula (I) wherein

A¹ is C—R⁵, A² is C—H, A³ is C—H and A⁴ is C—H;

G¹ is oxygen;

G² is C(R^{6a})(R^{6b}), oxygen, or N—R⁷;

R¹ is hydrogen;

R² is C₃-C₆alkyl or C₃-C₆alkyl substituted by halogen, C₄-C₆cycloalkyl, phenyl-C₁-C₂alkylene- or phenyl-C₁-C₂alkylene- wherein the phenyl moiety is substituted by one to five R¹⁰, pyridyl-C₁-C₂alkylene- or pyridyl-C₁-C₂alkylene- wherein the pyridyl moiety is substituted by one to four R¹⁰, oxetanyl, thietanyl, oxo-thietanyl, dioxo-thietanyl;

R³ is trifluoromethyl;

R⁴ is 3,5-dichloro-phenyl;

R⁵ is methyl;

R^{6a} is hydrogen;

R^{6b} is methyl or phenyl;

R⁷ is hydroxy;

R¹⁰ is bromo, chloro, fluoro or methyl.

For example, the present invention provides compounds of formula (I) wherein

A¹ is C—R⁵, A² is C—H, A³ is C—H and A⁴ is C—H;

G¹ is oxygen;

G² is C(R^{6a})(R^{6b}), oxygen, or N—R⁷;

R¹ is hydrogen;

R² is C₃-C₆alkyl or C₃-C₆alkyl substituted by halogen, pyridyl-methylene- or pyridyl-methylene- wherein the pyridyl moiety is substituted by one to three R¹⁰, thietanyl, oxo-thietanyl or dioxo-thietanyl.

R³ is trifluoromethyl;

R⁴ is 3,5-dichloro-phenyl;

R⁵ is methyl;

R^{6a} is hydrogen;

R^{6b} is methyl or phenyl;

R⁷ is hydroxy;

R¹⁰ is bromo, chloro, fluoro or methyl.

In one embodiment the present invention provides compounds of formula (Ia)

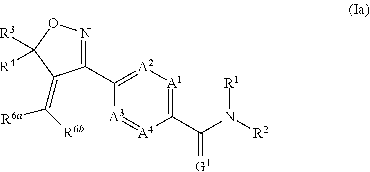

wherein A¹, A², A³, A⁴, G¹, R¹, R², R³, R⁴, R^{6a}, and R^{6b} are as defined for compounds of formula (I); or a salt or N-oxide thereof. The preferences for A¹, A², A³, A⁴, G¹, R¹, R², R³, R⁴, R^{6a} and R^{6b} are the same as the preferences set out for the corresponding substituents of compounds of the formula (I).

In another embodiment the present invention provides compounds of formula (Ib)

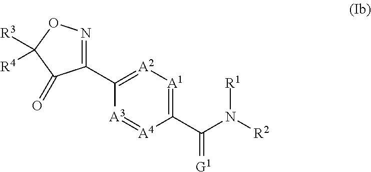

wherein A¹, A², A³, A⁴, G¹, R¹, R², R³ and R⁴ are as defined for compounds of formula (I); or a salt or N-oxide thereof. The preferences for A¹, A², A³, A⁴, G¹, R¹, R², R³ and R⁴ are the same as the preferences set out for the corresponding substituents of compounds of the formula (I).

In another embodiment the present invention provides compounds of formula (Ic)

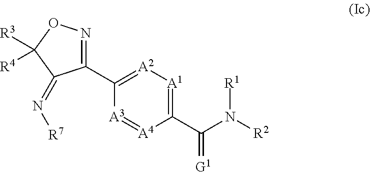

wherein A¹, A², A³, A⁴, G¹, R¹, R², R³, R⁴, and R⁷ are as defined for compounds of formula (I); or a salt or N-oxide thereof. The preferences for A¹, A², A³, A⁴, G¹, R¹, R², R³, R⁴ and R⁷ are the same as the preferences set out for the corresponding substituents of compounds of the formula (I).

Certain intermediates are novel and as such form a further aspect of the invention. One group of novel intermediates are compounds of formula (IIa)

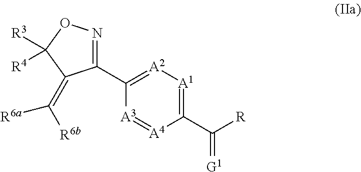

wherein A¹, A², A³, A⁴, G¹, R³, R⁴, R^{6a} and R^{6b} are as defined for a compound of formula (I), and R is hydroxy, C₁-C₆alkoxy or halogen, such as bromo, chloro or fluoro; or a salt or N-oxide thereof. The preferences for A¹, A², A³, A⁴, G¹, R³, R⁴, R^{6a} and R^{6b} are the same as the preferences set out for a compound of formula (I). Preferably R is hydroxy, C₁-C₆alkoxy or chloro.

Another group of novel intermediates are compounds of formula (IIa′)

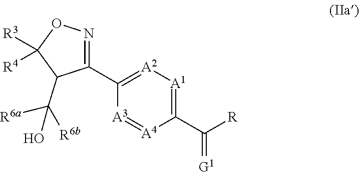

wherein A¹, A², A³, A⁴, G¹, R³, R⁴, R^{6a} and R^{6b} are as defined for a compound of formula (I), and R is as defined for a compound of formula (IIa); or a salt or N-oxide thereof. The preferences for A¹, A², A³, A⁴, G¹, R³, R⁴, R^{6a} and R^{6b} are the same as the preferences set out for a compound of formula (I). The preference for R is the same as the preference set out for a compound of formula (IIa).

Another group of novel intermediates are compounds of formula (IIb)

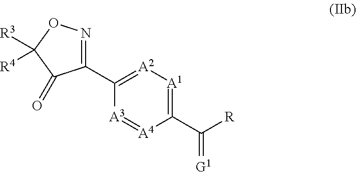

wherein A¹, A², A³, A⁴, G¹, R³ and R⁴ are as defined for a compound of formula (I), and R is hydroxy, C₁-C₆alkoxy or halogen, such as bromo, chloro or fluoro; or a salt or N-oxide thereof. The preferences for A¹, A², A³, A⁴, G¹, R³ and R⁴ are the same as the preferences set out for a compound of formula (I). The preference for R is the same as the preference set out for a compound of formula (IIa).

Another group of novel intermediates are compounds of formula (IIc)

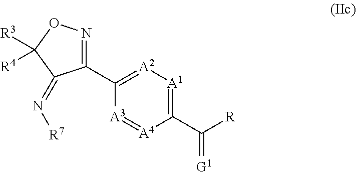

wherein A¹, A², A³, A⁴, G¹, R³, R⁴ and R⁷ are as defined for a compound of formula (I), and R is hydroxy, C₁-C₆alkoxy or halogen, such as bromo, chloro or fluoro; or a salt or N-oxide thereof. The preferences for A¹, A², A³, A⁴, G¹, R³, R⁴ and R⁷ are the same as the preferences set out for a compound of formula (I). The preference for R is the same as the preference set out for a compound of formula (IIa).

The compounds in Tables 1 to 5 below illustrate the compounds of the invention.

TABLE 1
Table 1 provides 51 compounds of formula (Ia′) wherein G¹ is oxygen,
R³ is trifluoromethyl, R⁴ is 3,5-dichloro-phenyl-,
R⁵ is methyl, R^{6a} is hydrogen, R^{6b} is methyl, and R¹ and R² have
the values listedin the table below.

| | |
|---|---|
| | (Ia′) |
| 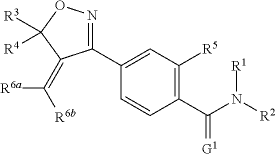 | |

| Compound | | |
|---|---|---|
| numbers | R¹ | R² |
| 1.01 | H | ethyl- |
| 1.02 | H | butyl- |
| 1.03 | H | but-2-yl- |
| 1.04 | H | 3-bromo-propyl- |
| 1.05 | H | 2,2,2-trifluoro-ethyl- |
| 1.06 | H | 3,3,3-trifluoro-propyl- |
| 1.07 | H | 2-methoxy-ethyl- |
| 1.08 | H | 1-methoxy-prop-2-yl- |
| 1.09 | H | cyclobutyl- |
| 1.10 | H | 2-methyl-cyclohex-1-yl- |
| 1.11 | H | phenyl-methyl- |
| 1.12 | H | 1-phenyl-eth-1-yl- |
| 1.13 | H | 2-phenyl-eth-1-yl- |
| 1.14 | H | (3-chloro-phenyl)-methyl- |
| 1.15 | H | (2-fluoro-phenyl)-methyl- |
| 1.16 | H | (4-methoxy-phenyl)-methyl- |
| 1.17 | H | (2-trifluoromethyl-phenyl)-methyl- |
| 1.18 | H | (2-trifluoromethoxy-phenyl)-methyl- |
| 1.19 | H | (pyrid-2-yl)-methyl- |
| 1.20 | H | (pyrid-3-yl)-methyl- |
| 1.21 | H | (2-chloro-pyrid-5-yl)-methyl- |
| 1.22 | H | (1-methyl-1H-imidazol-4-yl)-methyl- |
| 1.23 | H | (furan-2-yl)-methyl- |
| 1.24 | H | 2-(thiophen-2′-yl)-eth-1-yl- |
| 1.25 | H | 2-(indol-3′-yl)-eth-1-yl- |
| 1.26 | H | (1H-benzimidazol-2-yl)-methyl- |
| 1.27 | H | (oxetan-2-yl)-methyl- |
| 1.28 | H | (tetrahydrofuran-2-yl)-methyl- |
| 1.29 | H | 2-([1′,3′]dioxolan-2′-yl)-eth-1-yl- |
| 1.30 | H | 2-(morpholin-4′-yl)-eth-1-yl- |
| 1.31 | H | 2-(benzo[1′,3′]dioxol-5′-yl)-eth-1-yl- |
| 1.32 | H | (2,3-dihydro-benzo[1,4]dioxin-6-yl)-methyl- |
| 1.33 | H | 2-chloro-phenyl- |
| 1.34 | H | 3-fluoro-phenyl- |
| 1.35 | H | 2-methyl-phenyl- |
| 1.36 | H | 2-chloro-6-methyl-phenyl- |
| 1.37 | H | 2-trifluoromethyl-phenyl- |
| 1.38 | H | 2,4-dimethoxy-phenyl- |
| 1.39 | H | 3-methyl-pyrid-2-yl- |
| 1.40 | H | 1,3-dimethyl-1H-pyrazol-5-yl- |
| 1.41 | H | 4-methyl-thiazol-2-yl- |
| 1.42 | H | 5-methyl-thiadiazol-2-yl- |
| 1.43 | H | quinolin-2-yl- |
| 1.44 | H | quinolin-5-yl- |
| 1.45 | H | benzothiazol-6-yl- |
| 1.46 | H | 4-methyl-benzothiazol-2-yl- |
| 1.47 | H | thietan-3-yl- |
| 1.48 | H | 1-oxo-thietan-3-yl- |
| 1.49 | H | 1,1-dioxo-thietan-3-yl- |
| 1.50 | H | 3-methyl-thietan-3-yl- |
| 1.51 | H | N-(2,2,2-Trifluoro-ethyl)-acetamide-2-yl |

TABLE 2
Table 2 provides 51 compounds of formula (Ib′) wherein G¹ is oxygen,
R³ is trifluoromethyl, R⁴ is 3,5-dichloro-phenyl-,
R⁵ is methyl and R¹ and R² have the values listed in Table 1.

| | |
|---|---|
| | (Ib′) |
| 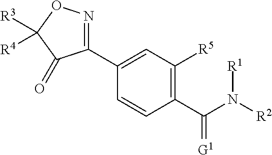 | |

TABLE 3
Table 3 provides 51 compounds of formula (Ic′) wherein G¹ is oxygen,
R³ is trifluoromethyl, R⁴ is 3,5-dichloro-phenyl-,
R⁵ is methyl, R⁷ is hydroxy, and R¹ and R² have the values listed
in Table 1.

| | |
|---|---|
| | (Ic′) |
| 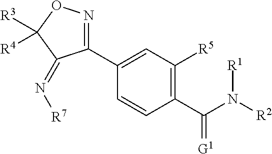 | |

TABLE 4
Table 4 provides 51 compounds of formula (Ic″) wherein G¹ is oxygen,
R³ is trifluoromethyl, R⁴ is 3,5-dichloro-phenyl-,
R⁵ is methyl, R⁷ is 2,2-difluoroethoxy, and R¹ and
R² have the values listed in Table 1.

| | |
|---|---|
| | (Ic″) |
| 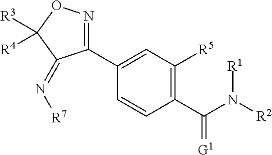 | |

TABLE 5
Table 5 provides 51 compounds of formula (Ic″′) wherein G¹ is oxygen,
R³ is trifluoromethyl, R⁴ is 3,5-dichloro-phenyl-,
R⁵ is methyl, R⁷ is methoxy, and R¹ and R² have
the values listed in Table 1.

| | |
|---|---|
| | (Ic″′) |
| 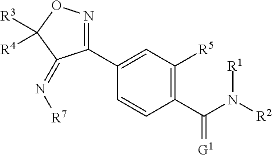 | |

The compounds of the invention may be made by a variety of methods as shown in Schemes 1 to 15.

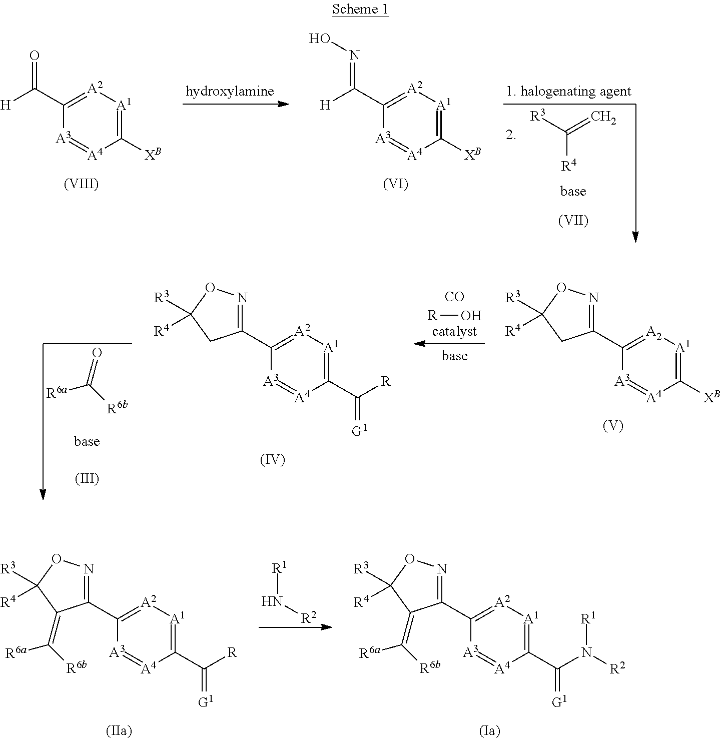

1) Compounds of formula (Ia), that is a compound of formula (I) wherein G² is C(R^{6a})(R^{6b}), wherein G¹ is oxygen, can be prepared by reacting a compound of formula (IIa), wherein G¹ is oxygen and R is OH, C₁-C₆alkoxy or Cl, F or Br, with an amine of formula HNR¹R² wherein R¹ and R² are as defined for a compound of formula (I) as shown in

### Scheme 1

When R is OH such reactions are usually carried out in the presence of a coupling reagent, such as N,N′-dicyclohexylcarbodiimide (“DCC”), 1-ethyl-3-(3-dimethyl-aminopropyl)carbodiimide hydrochloride (“EDC”) or bis(2-oxo-3-oxazolidinyl)phosphonic chloride (“BOP—Cl”), in the presence of a base, and optionally in the presence of a nucleophilic catalyst, such as 1-hydroxybenzotriazole (“HOBT”). When R is Cl, such reactions are usually carried out in the presence of a base, and optionally in the presence of a nucleophilic catalyst. Alternatively, it is possible to conduct the reaction in a biphasic system comprising an organic solvent, preferably ethyl acetate, and an aqueous solvent, preferably a solution of sodium hydrogen carbonate. When R is C₁-C₆alkoxy it is sometimes possible to convert the ester directly to the amide by heating the ester and amine together in a thermal process. Suitable bases include pyridine, triethylamine, 4-(dimethylamino)-pyridine (“DMAP”) or diisopropylethylamine (Hunig's base). Preferred solvents are N,N-dimethylacetamide, tetrahydrofuran, dioxane, 1,2-dimethoxyethane, ethyl acetate and toluene. The reaction is carried out at a temperature of from 0° C. to 100° C., preferably from 15° C. to 30° C., in particular at ambient temperature. Amines of formula HNR¹R² are commercially available or can be prepared using methods known to a person skilled in the art.

2) Acid halides of formula (IIa) wherein G¹ is oxygen and R is Cl, F or Br, may be made from carboxylic acids of formula (IIa) wherein G¹ is oxygen and R is OH, under standard conditions, such as treatment with thionyl chloride or oxalyl chloride. A preferred solvent is dichloromethane. The reaction is carried out at a temperature of from 0° C. to 100° C., preferably from 15° C. to 30° C., in particular at ambient temperature.

3) Carboxylic acids of formula (IIa) wherein G¹ is oxygen and R is OH, may be formed from esters of formula (IIa) wherein G¹ is oxygen and R is C₁-C₆alkoxy. It is known to a person skilled in the art that there are many methods for the hydrolysis of such esters depending on the nature of the alkoxy group. One widely used method to achieve such a transformation is the treatment of the ester with an alkali hydroxide, such as lithium hydroxide, sodium hydroxide or potassium hydroxide, in a solvent, such as ethanol or tetrahydrofuran, in the presence of water. Another transformation is the treatment of the ester with an acid, such as trifluoroacetic acid, in a solvent, such as dichloromethane, followed by addition of water. The reaction is carried out at a temperature of from 0° C. to 150° C., preferably from 15° C. to 100° C., in particular at 50° C.

4) Where R^{ha} and R^{6b} are independently of each other hydrogen, C₁-C₈alkyl or C₁-C₈alkyl substituted by one to five R¹², C₂-C₈alkenyl or C₂-C₈alkenyl substituted by one to five R¹², C₂-C₈alkynyl, C₂-C₈haloalkynyl, aryl or aryl substituted by one to five R¹³, or heterocyclyl or heterocyclyl substituted by one to five R¹³, compounds of formula (IIa), wherein G¹ and R are as defined under 1), can be made by reaction of a compound of formula (IV) wherein G¹ and R are as defined under 1), with base, such as lithium diisopropylamine (“LDA”), followed by the addition of an aldehyde or a ketone of formula (III) wherein R^{6a} and R^{6b} are as defined above. The reaction is carried out at a temperature of from −120° C. to +30° C., preferably from −100° C. to 0° C. This reaction may afford a compound of formula (IIa) directly or alternatively an intermediate of formula (IIa′)

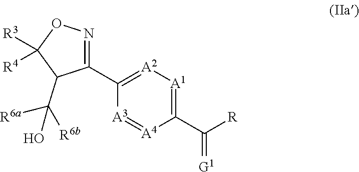

The intermediate of formula (IIa′) can be transformed into a compound of formula (IIa) by reaction with a base, such as lithium hydroxide, in a solvent, such as tetrahydrofuran or water or a mixture thereof, at a temperature of from 0° C. to 100° C., preferably at ambient temperature. It can also be transformed into compound of formula (IIa) by dehydration. Such reactions are usually carried out in the presence of an acid, for example an inorganic acid, such as hydrochloric acid or sulfuric acid, or a sulfonic acid, such as methanesulfonic acid, optionally in a solvent, such as water, ethanol or tetrahydrofuran, or mixtures thereof. The reaction is carried out at a temperature of from 0° C. to 100° C., preferably from 40° C. to 80° C. Representative experimental conditions for this transformation are described in Synthetic Communications 2003, 23, 4163-4171. Alternatively, dehydration can be carried out using a dehydrating agent, such as phosphorus pentoxide, Burgess' dehydrating agent (1-methoxy-N-triethylammoniosulfonyl-methanimidate) or Martin's sulfurane (Bis[α,α-bis(trifluoromethyl)benzyloxy]diphenylsulfur), in a solvent, such as chloroform or dichloromethane, at a temperature of from −20° C. to 50°, preferably at 0° C.

5) Compounds of formula (IV) wherein G¹ is oxygen and R is C₁-C₆alkoxy, can be prepared by reacting a compound of formula (V) wherein X^{B} is a leaving group, for example a halogen, such as bromo, with carbon monoxide and an alcohol of formula R—OH, such as ethanol, in the presence of a catalyst, such as bis(triphenylphosphine)palladium(II) dichloride, and a base, such as pyridine, triethylamine, 4-(dimethylamino)-pyridine (“DMAP”) or diisopropylethylamine (Hunig's base). The reaction is carried out at a temperature of from 50° C. to 200° C., preferably from 100° C. to 150° C., in particular at 115° C. The reaction is carried out at a pressure of from 50 to 200 bar, preferably from 100 to 150 bar, in particular at 120 bar.

6) Compounds of formula (V) wherein X^{B} is as defined under 5), can be made by reaction of an oxime of formula (VI) wherein X^{B} is as defined under 5), and a vinyl compound of formula (VII) in a two step reaction. In the first step, the oxime of formula (VI) is reacted with a halogenating agent, for example a succinimide, such as N-chloro-succinimide (“NCS”), in the presence of a suitable solvent, for example a polar solvent, such as N,N-dimethylformamide. The first step is carried out at a temperature of from 0° C. to 100° C., preferably from 15° C. to 30° C., in particular at ambient temperature.

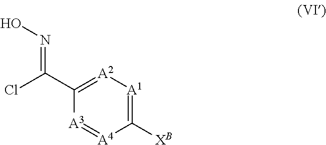

In the second step, the chloro hydroxy imine intermediate of formula (VI′) is reacted with the vinyl compound of formula (VII) in the presence of a base, for example an organic base, such as triethylamine, or an inorganic base, such as sodium hydrogen carbonate, in the presence of a suitable solvent, for example a polar solvent, such as N,N-dimethylformamide or isopropanol. It is possible to conduct these two steps separately and optionally to isolate the chloro hydroxy imine intermediate or more conveniently to conduct these two steps successively in one reaction vessel without isolation of the intermediate. The second step is carried out at a temperature of from 0° C. to 100° C., preferably from 15° C. to 30° C., in particular at ambient temperature. Vinyl compounds of formula (VII) are commercially available or can be made by methods known to a person skilled in the art.

7) Compounds of formula (VI) wherein X^{B} is as defined under 5), can be made by reaction of an aldehyde of formula (VIII) wherein X^{B} is as defined under 5), with a hydroxylamine, such as hydroxylamine hydrochloride. Such reactions are carried out optionally in the presence of a base, for example an organic base, such as triethylamine or sodium acetate, or an inorganic base, such as sodium hydrogen carbonate, optionally in the presence of a solvent, for example an alcohol, such as methanol or ethanol, or water, or mixtures thereof. The reaction is carried out at a temperature of from 0° C. to 100° C., preferably from 15° C. to 30° C., in particular at ambient temperature. Aldehydes of formula (VIII) are commercially available or can be made by methods known to a person skilled in the art.

8) Compounds of formula (Ia) as defined under 1) wherein G¹ is sulfur, may be made by treatment of a compound of formula (IIa) as defined under 1) wherein G¹ and R are as defined under 1), with a thio-transfer reagent such as Lawesson's reagent or phosphorus pentasulfide prior to elaborating to compounds of formula (Ia), as described under 1).

9) Compounds of formula (I) with a sulfoxide group or a sulfone group can be made from a compound of formula (I) with a sulfide group (or sulfoxide group) in the corresponding position, by treatment with an oxidising reagent, such as potassium permanganate, 3-chloroperoxybenzoic acid (“MCPBA”), sodium periodate (optionally in the presence of ruthenium (II) oxide), hydrogen peroxide, oxone and sodium hypochlorite. One equivalent of oxidising reagent is required to convert a sulfide to a sulfoxide, or a sulfoxide to a sulfone. Two equivalents of oxidising reagent are required to convert a sulfide to a sulfone. Preferred solvents are tetrahydrofuran, dioxane, 1,2-dimethoxyethane, ethyl acetate, toluene, dichloromethane and water, or mixtures thereof. The reaction is optionally carried out in the presence of a base, for example a carbonate, such as sodium hydrogen carbonate. The reaction is carried out at a temperature of from 0° C. to 100° C., preferably from 15° C. to 30° C., in particular at ambient temperature.

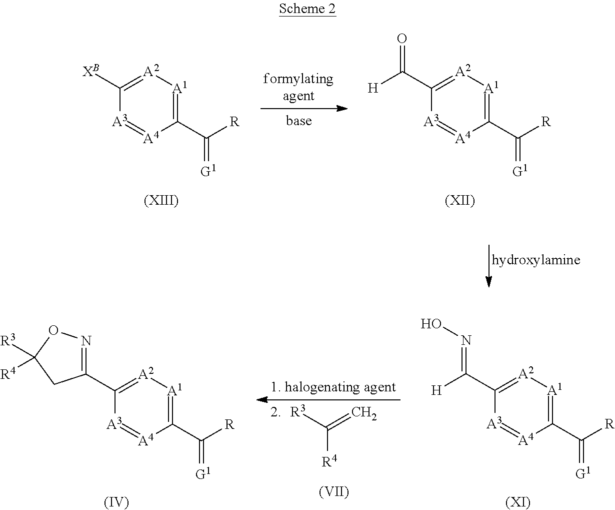

10) Alternatively, compounds of formula (IV) wherein G¹ is oxygen and R is C₁-C₆alkoxy, such as methoxy or tert-butoxy, can be prepared by reaction of an oxime of formula (XI) wherein G¹ is oxygen and R is C₁-C₆alkoxy, such as methoxy or tert-butoxy, with a halogenating agent followed by a vinyl compound of formula (VII) and base in a two step reaction as described under 6). The intermediate of formula (XI′) wherein G¹ is oxygen and R is C₁-C₆alkoxy, such as methoxy or tert-butoxy, can optionally be isolated.

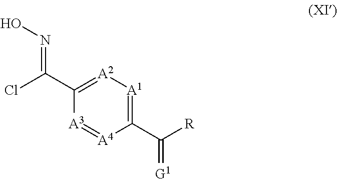

11) Compounds of formula (XI) wherein G¹ and R are as defined under 10), can be made by reaction of an aldehyde of formula (XII) wherein G¹ and R are as defined under 10), with a hydroxylamine and optionally a base as described under 7).

12) Compounds of formula (XII) wherein G¹ and R are as defined under 10), can be prepared by reaction of a compound of formula (XIII) wherein G¹ and R are as defined under 10), and X^{B} is as defined under 5), with a formylating agent, such as N,N-dimethyl-formamide. Such reactions are carried out in the presence of a base, for example a lithium base, such as butyl lithium, in the presence of a suitable solvent, for example a polar solvent, such as tetrahydrofuran or excess N,N-dimethylformamide. Compounds of formula (XIII) wherein G¹ and R are as defined under 10), are commercially available or can be made by methods known to a person skilled in the art.

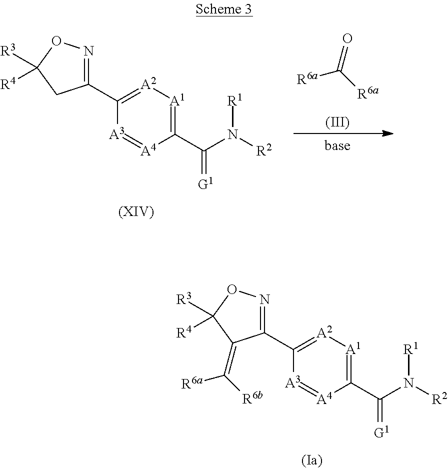

13) Alternatively, compounds of formula (Ia) as defined under 1) can be prepared by reaction of a compound of formula (XIV) wherein G¹ is oxygen, with a base and an aldehyde or a ketone of formula (III) as shown in Scheme 3 using the method described under 4). This reaction may afford a compound of formula (Ia) directly or alternatively an intermediate of formula (Ia′)

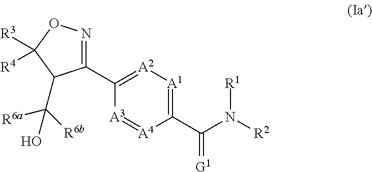

The intermediate of formula (Ia′) can be transformed into a compound of formula (Ia) by reaction with a base as described under 4). Compounds of formula (XIV) wherein G¹ is oxygen, can be made by methods described, for example, in EP 1,731,512.

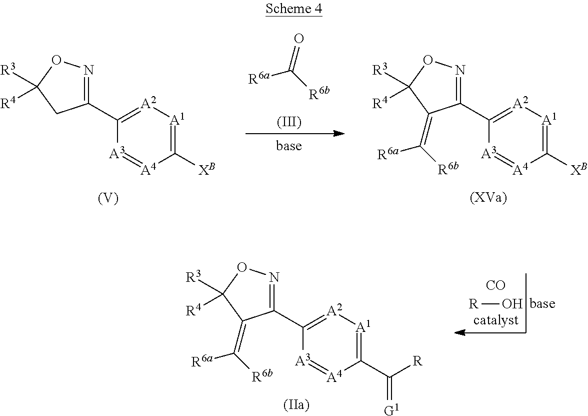

14) Alternatively, compounds of formula (IIa) as defined under 1) can be obtained from a compound of formula (XVa), wherein G¹ is oxygen and X^{B} is as defined under 5), as shown in Scheme 4 using the method described under 5).

15) Compounds of formula (XVa) as defined under 14) can be made from a compound of formula (V) as described under 4). This reaction may afford a compound of formula (XVa) directly or alternatively an intermediate of formula (XVa′)

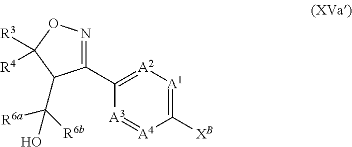

The intermediate of formula (XVa′) can be transformed into a compound of formula (XVa) by reaction with a base as described under 4).

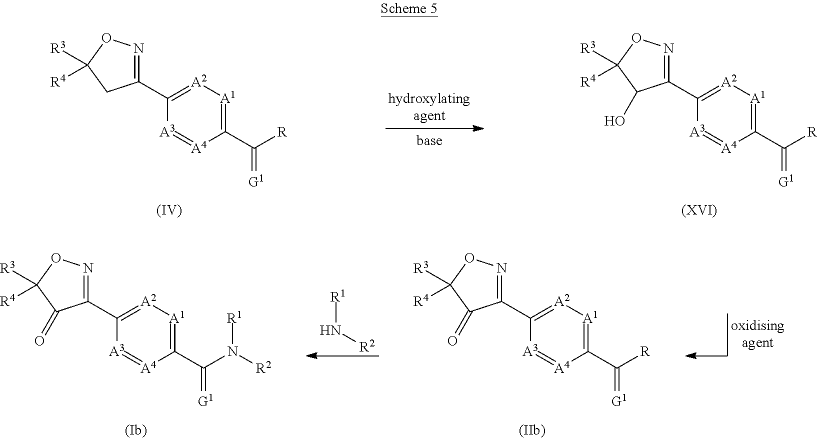

16) Compounds of formula (Ib), that is a compound of formula (I) wherein G² is oxygen, wherein G¹ is oxygen, can be prepared by reacting a compound of formula (IIb), wherein G¹ and R are as defined under 1), with an amine of formula HNR¹R² as shown in Scheme 5 using the methods described under 1).

17) Compounds of formula (Ith) as defined under 16) can be prepared by reacting an alcohol of formula (XVI) wherein G¹ and R are as defined under 1), with an oxidising agent, such as chromium trioxide, manganese dioxide or pyridinium chlorochromate.

18) Compounds of formula (XVI) wherein G¹ and R are as defined under 1), can be made by reaction of a compound of formula (IV) wherein G¹ and R are as defined under 1), with a base, such as lithium diisopropylamine (“LDA”), followed by the addition of an electrophilic hydroxylating agent, such as oxaziridines (for instance, N-sulfonyl oxaziridines), oxodiperoxymolybdenum(pyridine)-(hexamethylphosphoric triamide) (“MoOPH”) or a peroxide (for instance, bis(trimethylsilyl)peroxide). The reaction is carried out at a temperature of from −120° C. to +30° C., preferably from −100° C. to 0° C.

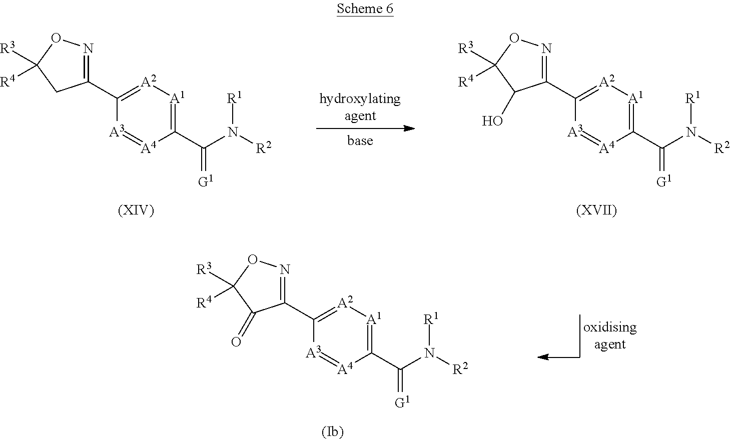

19) Alternatively, compounds of formula (Ib) as defined under 16) can be prepared by reaction of an alcohol of formula (XVII) wherein G¹ is oxygen, with an oxidising agent as shown in Scheme 6 using the method described under 17).

20) Compounds of formula (XVII) wherein G¹ is oxygen, can be prepared by reaction of a compound of formula (XIV) wherein G¹ is oxygen, with a base and an electrophilic hydroxylating agent as described under 18).

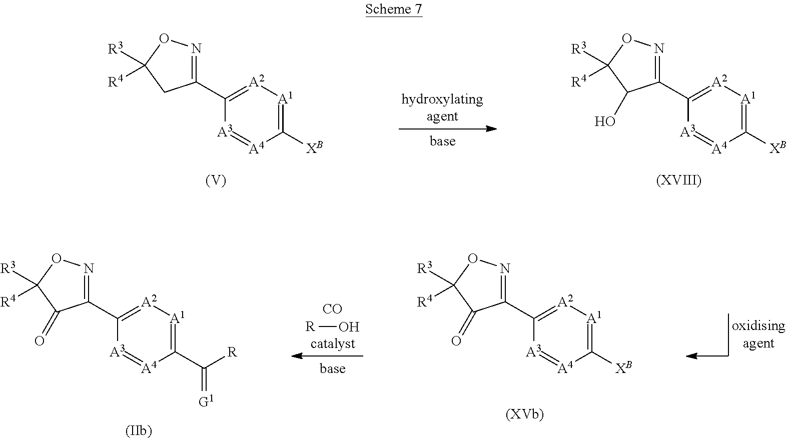

21) Alternatively, compounds of formula (IIb) as defined under 16) may be obtained from a compound of formula (XVb), wherein X^{B} is as defined under 5), as shown on Scheme 7 using the method described under 5).

22) Compounds of formula (XVb) can be prepared by reaction of a compound of formula (XVIII) with an oxidizing agent as described under 17).

23) Compounds of formula (XVIII) can be prepared by reaction of a compound of formula (V) with a base and an electrophilic hydroxylating agent as described under 18).

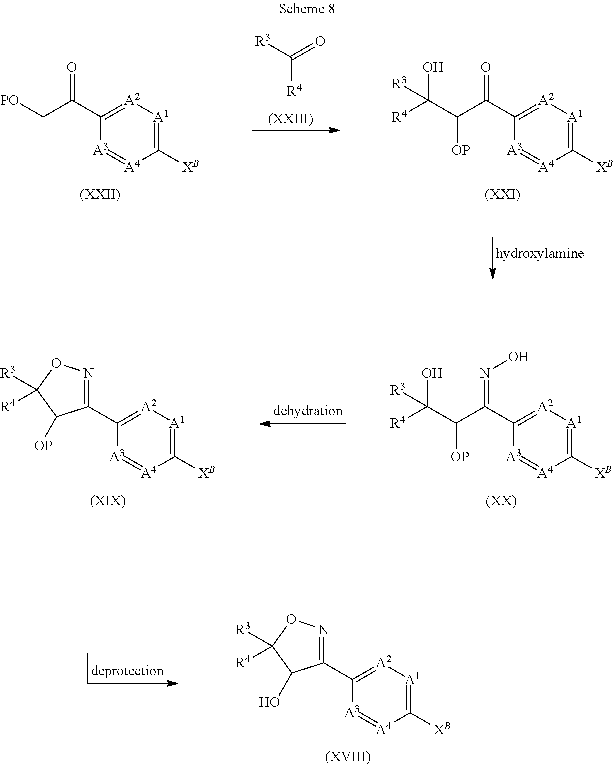

24) Alternatively, compounds of formula (XVIII) can be obtained after deprotection in of a compound of formula (XIX), wherein P is a protecting group, such as benzoyl or trialkylsilyl as shown in Scheme 8. Various methods known to the skilled person exist for such reactions, examples can be found in Greene, T. W., Wuts, P. G. N., Protective Groups in Organic Synthesis, John Wiley & Sons, Inc, 2006.

25) Compounds of formula (XIX) wherein P is as defined under 24) and X^{B} is as defined under 5), can be prepared by cyclisation of a compound of formula (XX) wherein X^{B} is as defined under 5). The cyclisation of a compound of formula (XX) can also be referred to as the dehydration of a compound of formula (XX). Such reactions are usually carried out in the presence of an acid, for example an inorganic acid, such as hydrochloric acid or sulfuric acid, or a sulfonic acid, such as methanesulfonic acid, optionally in a solvent, such as water, ethanol or tetrahydrofuran, or mixtures thereof. The reaction is carried out at a temperature of from 0° C. to 100° C., preferably from 40° C. to 80° C. Representative experimental conditions for this transformation are described in Synthetic Communications 2003, 23, 4163-4171. Alternatively, dehydration can be carried out using a dehydrating agent, such as phosphorus pentoxide, in a solvent, such as chloroform, at a temperature of from −20° C. to 50° C., preferably at 0° C., as described in Journal of Heterocyclic Chemistry 1990, 27, 275. Alternatively, cyclisation can be carried out under Mitsunobu conditions involving treatment of a compound of formula (XX) with a phosphine, such as triphenylphosphine, and an azodicarboxylate reagent, such as diethyl azodicarboxylate, diisopropyl azodicarboxylate or dicyclohexyl azodicarboxylate, in a solvent, such as tetrahydrofuran, at a temperature of from 0° C. to 80° C., preferably from 0° C. to ambient temperature.

26) Compounds of formula (XX) wherein P is as defined under 24) and X^{B} is as defined under 5), can be made by reaction of a β-hydroxy ketone of formula (XXI) wherein P is as defined under 24) and X^{B} is as defined under 5), with a hydroxylamine and optionally a base as described under 7).

27) Compounds of formula (XXI) wherein P is as defined under 24) and X^{B} is as defined under 5) can be made by aldol reaction of a substituted methyl ketone of formula (XXII) wherein P is as defined under 24) and X^{B} is as defined under 5) with a ketone of formula (XXIII) wherein R³ and R⁴ are as defined for a compound of formula (I). Such reactions are usually carried out in the presence of a base, such as sodium hydride, lithium hydride, lithium diisopropylamide or lithium hexamethyldisilazide, in a solvent, such as tetrahydrofuran, at a temperature of from −78° C. to +100° C., preferably from 0° C. to +80° C. Alternatively, the reaction can be performed using a Lewis acid, such as titanium tetrachloride or tin bis(trifluoromethanesulfonate) and optionally an amine, such as triethylamine, diisopropylethylamine, tetramethylethylenediamine (“TMEDA”) or tributylamine, in a solvent, such as dichloromethane, at a temperature of from −78° C. to ambient temperature, preferably at −78° C. Representative conditions for such a transformation are given in Tetrahedron, 58(41), 8269-8280; 2002 or Tetrahedron, 40(8), 1381-90; 1984. Ketones of formula (XXIII) are commercially available or can be made by methods known to a person skilled in the art. Substituted methyl ketones of formula (XXII) are known in the literature or can be prepared, for example, from the corresponding unsubstituted ketone.

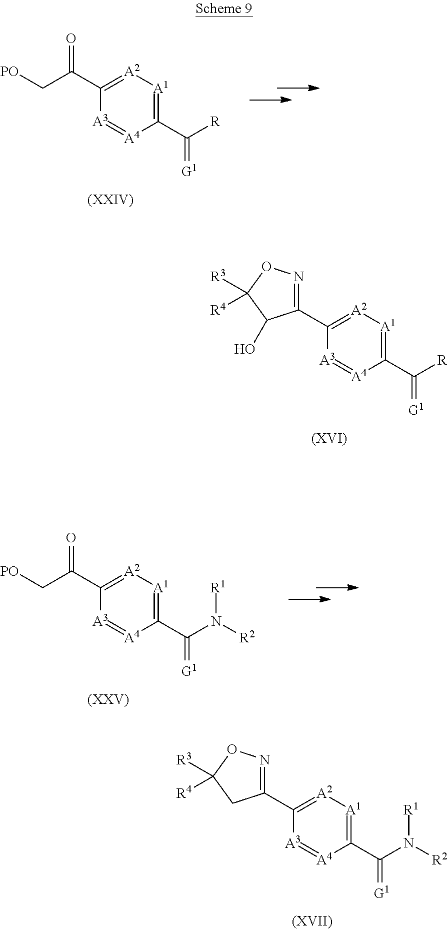

28) Similarly, compounds of formula (XVI) wherein G¹ and R are as defined under 1), and compounds of formula (XVII) wherein G¹ is oxygen can be made from a compound of formula (XXIV) wherein G¹ is oxygen, P is as defined under 24) and R is as defined under 1), and a compound of formula (XXV) wherein G¹ is oxygen and P is as defined under 24), respectively, as shown in Scheme 9 using a similar sequence to that shown in Scheme 8 and using the methods described under 24), 25), 26) and 27).

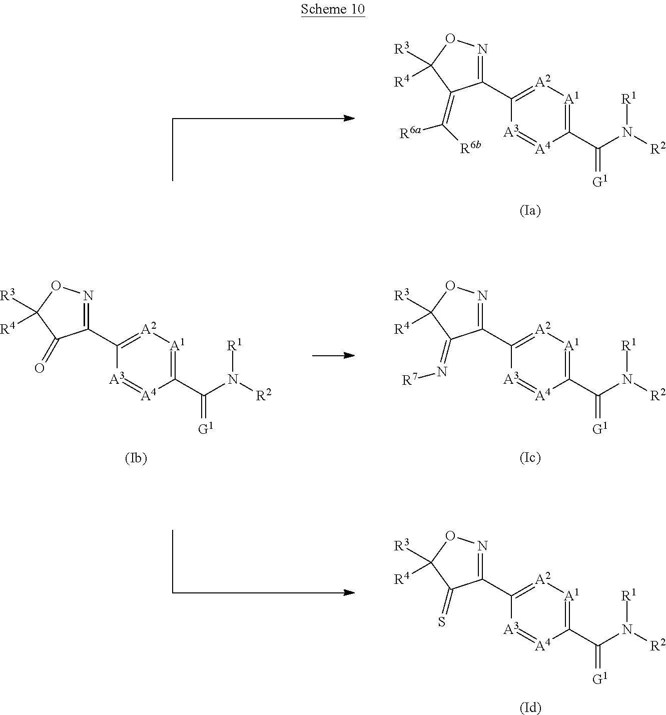

29) Compounds of formula (Ib) as defined under 16) can be further converted into compounds of formula (I) as shown in Scheme 10. For example, where R^{6a} and R^{6b} are independently of each other hydrogen, C₁-C₈alkyl or C₁-C₈alkyl substituted by one to five R¹², C₂-C₈alkenyl or C₂-C₈alkenyl substituted by one to five R¹², C₂-C₈alkynyl, C₂-C₈haloalkynyl, C₁-C₈alkoxy-, C₁-C₈haloalkoxy-, C₁-C₈alkylcarbonyl-, C₁-C₈alkoxycarbonyl-, aryl or aryl substituted by one to five R¹³, or heterocyclyl or heterocyclyl substituted by one to five R¹³, or NR¹⁴R¹⁵, wherein R¹⁴ and R¹⁵ are independently hydrogen, C₁-C₈alkyl, C₁-C₈haloalkyl, C₁-C₈alkylcarbonyl-, C₁-C₈alkoxycarbonyl-, or R¹⁴ and R¹⁵ together with the nitrogen atom to which they are attached form a 3 to 7 membered heterocyclic ring; or R^{6a} and R^{6b} together with the carbon atom to which they are attached form a 3 to 7 membered carbocyclic or heterocyclic ring, compounds of formula (Ia) as defined under 1) may be obtained by a methylenation reaction such as a Wittig reaction of a ketone of formula (Ib) with a phosphonium glide of formula Ph₃P═C(R^{6a})(R^{6b}), or Horner- Wadsworth-Emmons) reaction of a ketone of formula (Ib) with a phosphonate of formula (OEt)₂P(O)CH(R^{6a})(R^{6b}) and a base, such as sodium hydride, or by reaction with Tebbe's reagent (Bis(cyclopentadienyl)-μ-chloro-(dimethylaluminum)-μ-methylenetitanium). Such reactions are known to a person skilled in the art. In another example, where R^{6a} and R^{6b} are independently hydrogen or halogen provided that at least one of R^{6a} and/or R^{6b} is halogen, compounds of formula (Ia) as defined under 1) may be obtained by reaction of a ketone of formula (Ib) with a tetrachloromethane, tetrabromomethane or dibromodifluoromethane in the presence of a phosphine, such as triphenylphosphine. Such reactions are known to a person skilled in the art. In another transformation, compounds of formula (Ic), that is a compound of formula (I) wherein G² is N—R⁷, may be obtained by condensation of a ketone of formula (Ib) with an amine of formula R⁷NH₂ wherein R⁷ is as defined for a compound of formula (I). Such reactions are known to a person skilled in the art. In another transformation, compounds of formula (Id), that is a compound of formula (I) wherein G² is sulfur, may be obtained by treatment of a ketone of formula (Ib) with a thio-transfer reagent such as Lawesson's reagent or phosphorus pentasulfide. Such reactions are known to a person skilled in the art.

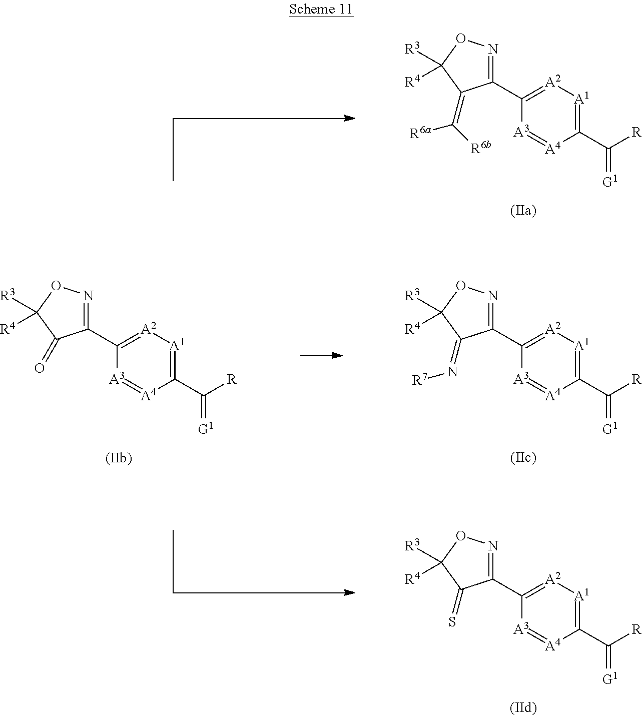

30) Similarly, compounds of formula (IIa) wherein G¹ and R are as defined under 1), compounds of formula (Ith) wherein G¹ and R are as defined under 1), and compounds of formula (IId) wherein G¹ and R are as defined under 1), may be obtained from a ketone of formula (Ith) wherein G¹ and R are as defined under 1), as shown in Scheme 11 using the methods described under 29).

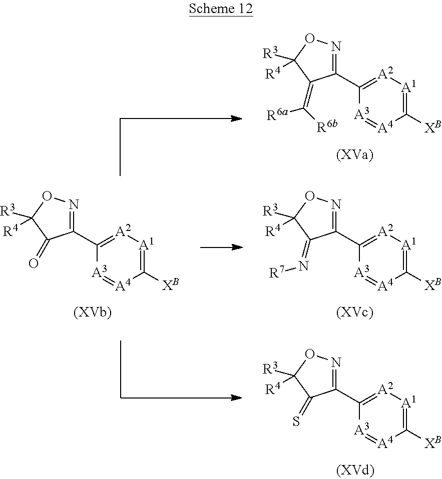

31) Similarly, compounds of formula (XVa) wherein X^{B} is as defined under 5), compounds of formula (XVb) wherein X^{B} is as defined under 5) and compounds of formula (XVd) wherein X^{B} is as defined under 5) may be obtained from a ketone of formula (XVb) wherein X^{B} is as defined under 5) as shown in Scheme 12 using the methods described under 29).

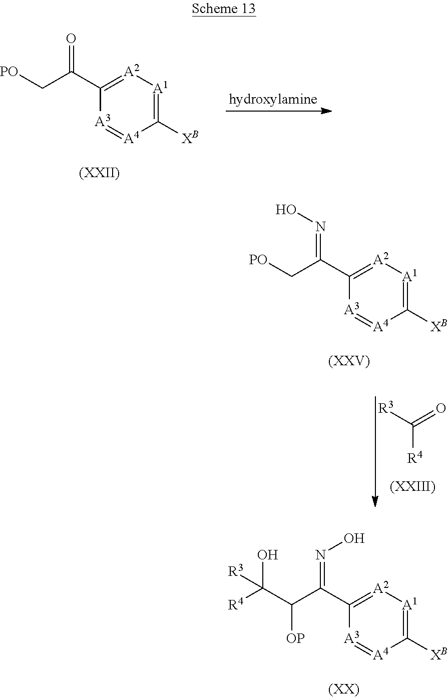

32) Alternatively, compounds of formula (XX) wherein X^{B} is as defined under 5), can be prepared by reacting a substituted methyl oxime of formula (XXV) wherein P is as defined under 24) and X^{B} is as defined under 5), with a ketone of formula (XXIII) wherein R³ and R⁴ are as defined for a compound of formula (I) in an aldol-type reaction as shown in Scheme 13. Such reactions are usually carried out by treating the substituted methyl oxime in of formula (XXV) with a base, such as butyl lithium, lithium diisopropylamide or lithium hexamethyldisilazide, in a solvent, such as tetrahydrofuran, at a temperature of from −78° C. to ambient temperature, preferably from −20° C. to 0° C., followed by addition of the ketone of formula (XXIII) at a temperature of from −78° C. to 0° C., preferably at 0° C. Representative conditions for such a transformation can be found in Synthetic Communications, 2003, 23, 4163-4171.

33) Compounds of formula (XXV) wherein P is as defined under 24) and X^{B} is as defined under 5), can be made by reaction of a substituted methyl ketone of formula (XXII) wherein P is as defined under 24) and X^{B} is as defined under 5), with a hydroxylamine and optionally a base as described under 7).

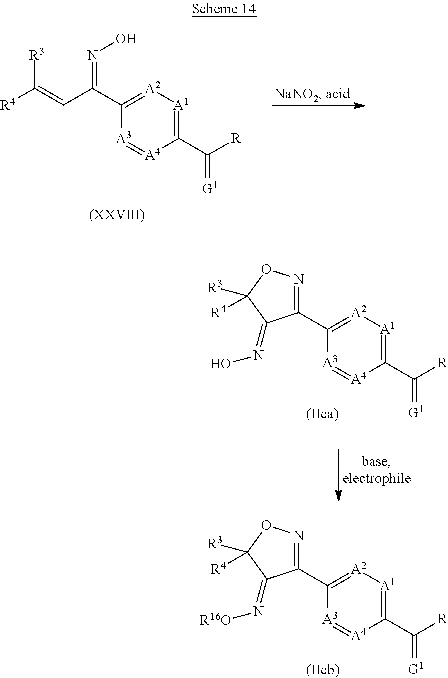

34) Alternatively, where R¹⁶ is independently halogen, cyano, nitro, hydroxy, C₁-C₈alkoxy-, C₁-C₈haloalkoxy-, C₁-C₈alkylcarbonyl-, C₁-C₈alkoxycarbonyl-, mercapto, C₁-C₈alkylthio-, C₁-C₈haloalkylthio-, C₁-C₈alkylsulfinyl-, C₁-C₈haloalkylsulfinyl-, C₁-C₈alkylsulfonyl-, or C₁-C₈haloalkylsulfonyl-, a compound of formula (IIcb), that is a compound of formula (II) wherein G² is N—OR¹⁶, wherein G¹ and R are as defined under 1), may be obtained by reaction of a compound of formula (IIca) wherein G¹ and R are as defined under 1), with base, such as sodium hydride, followed by the addition of an electrophile, for example an alkylating agent, an acylating agent or a sulfonylating agent, in the presence of a suitable solvent, such as tetrahydrofuran or dimethylformamide, as shown in Scheme 14. Suitable alkylating agents are, for example, alkyl halides, such as methyl iodide (Me-I), for making a compound where R¹⁶ is C₁-C₈alkyl, in particular methyl. The reaction is carried out at a temperature of from −120° C. to +30° C., preferably from −100° C. to 0° C.

35) Alternatively, a compound of formula (IIca), that is a compound of formula (II) wherein G² is N—OH, wherein G¹ and R are as defined under 1), may be obtained by reaction of a compound of formula (XXVIII) wherein G¹ and R are as defined under 1), with sodium nitrite followed by an acid such as hydrochloric acid. An example of such a transformation can be found in Russian Chemical Bulletin (2006), 55(3), 535-542. Compounds of formula (XVIII) can be prepared by a number of ways, for example as described in WO 09/001,942.

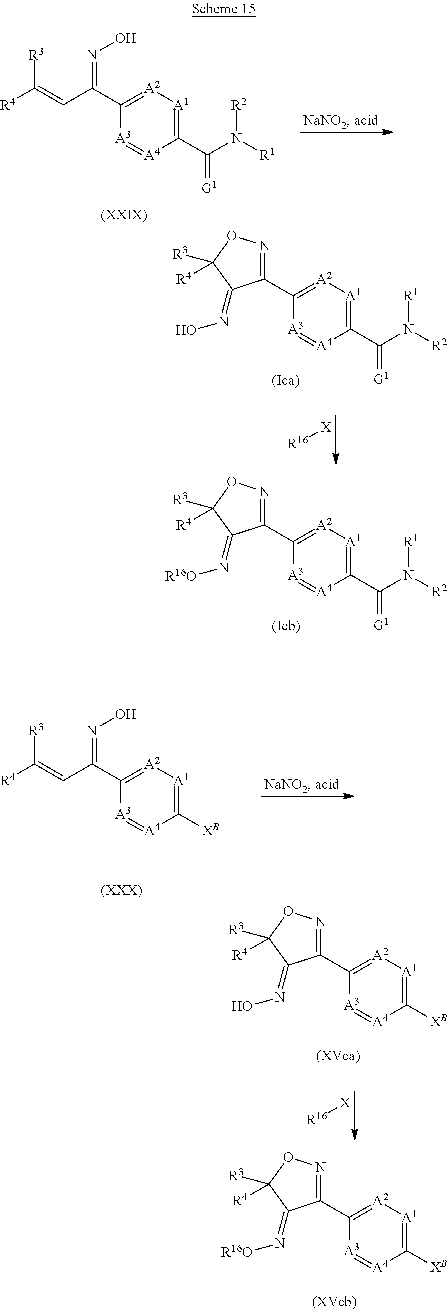

36) Similarly, compounds of formula (Icb) wherein G¹ is oxygen and compounds of formula (XVcb) wherein X^{B} is as defined under 5) can be prepared from a compound of formula (Ica) wherein G¹ is oxygen and a compound of formula (XVca) wherein X^{B} is as defined under 5), respectively, as shown in Scheme 15 using the method described under 35).

37) Similarly, compounds of formula (Ica) wherein G¹ is oxygen and compounds of formula (XVca) wherein X^{B} is as defined under 5) can be prepared from a compound of formula (XXIX) wherein G¹ is oxygen and a compound of formula (XXX) wherein X^{B} is as defined under 5), respectively, as shown in Scheme 15 using the method described under 36).

The compounds of formula (I) can be used to combat and control infestations of insect pests such as Lepidoptera, Diptera, Hemiptera, Thysanoptera, Orthoptera, Dictyoptera, Coleoptera, Siphonaptera, Hymenoptera and Isoptera and also other invertebrate pests, for example, acarine, nematode and mollusc pests. Insects, acarines, nematodes and molluscs are hereinafter collectively referred to as pests. The pests which may be combated and controlled by the use of the invention compounds include those pests associated with agriculture (which term includes the growing of crops for food and fiber products), horticulture and animal husbandry, companion animals, forestry and the storage of products of vegetable origin (such as fruit, grain and timber); those pests associated with the damage of man-made structures and the transmission of diseases of man and animals; and also nuisance pests (such as flies).

The compounds of the invention may be used for example on turf, ornamentals, such as flowers, shrubs, broad-leaved trees or evergreens, for example conifers, as well as for tree injection, pest management and the like.

Examples of pest species which may be controlled by the compounds of formula (I) include: Myzus persicae (aphid), Aphis gossypii (aphid), Aphis fabae (aphid), Lygus spp. (capsids), Dysdercus spp. (capsids), Nilaparvata lugens (planthopper), Nephotettixc incticeps (leafhopper), Nezara spp. (stinkbugs), Euschistus spp. (stinkbugs), Leptocorisa spp. (stinkbugs), Frankliniella occidentalis (thrip), Thrips spp. (thrips), Leptinotarsa decemlineata (Colorado potato beetle), Anthonomus grandis (boll weevil), Aonidiella spp. (scale insects), Trialeurodes spp. (white flies), Bemisia tabaci (white fly), Ostrinia nubilalis (European corn borer), Spodoptera littoralis (cotton leafworm), Heliothis virescens (tobacco budworm), Helicoverpa armigera (cotton bollworm), Helicoverpa zea (cotton bollworm), Sylepta derogata (cotton leaf roller), Pieris brassicae (white butterfly), Plutella xylostella (diamond back moth), Agrotis spp. (cutworms), Chilo suppressalis (rice stem borer), Locusta. migratoria (locust), Chortiocetes terminifera (locust), Diabrotica spp. (rootworms), Panonychus ulmi (European red mite), Panonychus citri (citrus red mite), Tetranychus urticae (two-spotted spider mite), Tetranychus cinnabarinus (carmine spider mite), Phyllocoptruta oleivora (citrus rust mite), Polyphagotarsonemus latus (broad mite), Brevipalpus spp. (flat mites), Boophilus microplus (cattle tick), Dermacentor variabilis (American dog tick), Ctenocephalides fells (cat flea), Liriomyza spp. (leafminer), Musca domestica (housefly), Aedes aegypti (mosquito), Anopheles spp. (mosquitoes), Culex spp. (mosquitoes), Lucillia spp. (blowflies), Blattella germanica (cockroach), Periplaneta americana (cockroach), Blatta orientalis (cockroach), termites of the Mastotermitidae (for example Mastotermes spp.), the Kalotermitidae (for example Neotermes spp.), the Rhinotermitidae (for example Coptotermes formosanus, Reticulitermes flavipes, R. speratu, R. virginicus, R. hesperus, and R. santonensis) and the Termitidae (for example Globitermes sulfureus), Solenopsis geminata (fire ant), Monomorium pharaonis (pharaoh's ant), Damalinia spp. and Linognathus spp. (biting and sucking lice), Meloidogyne spp. (root knot nematodes), Globodera spp. and Heterodera spp. (cyst nematodes), Pratylenchus spp. (lesion nematodes), Rhodopholus spp. (banana burrowing nematodes), Tylenchulus spp.(citrus nematodes), Haemonchus contortus (barber pole worm), Caenorhabditis elegans_(vinegar eelworm), Trichostrongylus spp. (gastro intestinal nematodes) and Deroceras reticulatum (slug).

The invention therefore provides a method of combating and controlling insects, acarines, nematodes or molluscs which comprises applying an insecticidally, acaricidally, nematicidally or molluscicidally effective amount of a compound of formula (I), or a composition containing a compound of formula (I), to a pest, a locus of pest, preferably a plant, or to a plant susceptible to attack by a pest, The compounds of formula (I) are preferably used against insects, acarines or nematodes.

The invention provides a method of combating and/or controlling insects, acarines, nematodes or molluscs which comprises applying an insecticidally, acaricidally, nematicidally or molluscicidally effective amount of a compound of formula (I), or a composition containing a compound of formula (I), to a pest, a locus of pest, preferably a plant, or to a plant susceptible to attack by a pest, The compounds of formula (I) are preferably used against insects, acarines or nematodes.

The term “plant” as used herein includes seedlings, bushes and trees.

Crops are to be understood as also including those crops which have been rendered tolerant to herbicides or classes of herbicides (e.g. ALS-, GS-, EPSPS-, PPO- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®.

Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood as being those which are obtained by conventional methods of breeding or genetic engineering and contain so-called output traits (e.g. improved storage stability, higher nutritional value and improved flavor).

In order to apply a compound of formula (I) as an insecticide, acaricide, nematicide or molluscicide to a pest, a locus of pest, or to a plant susceptible to attack by a pest, a compound of formula (I) is usually formulated into a composition which includes, in addition to the compound of formula (I), a suitable inert diluent or carrier and, optionally, a surface active agent (SFA). SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a compound of formula (I). The composition is generally used for the control of pests such that a compound of formula (I) is applied at a rate of from 0.1 g to 10 kg per hectare, preferably from 1 g to 6 kg per hectare, more preferably from 1 g to 1 kg per hectare.

When used in a seed dressing, a compound of formula (I) is used at a rate of 0.0001 g to 10 g (for example 0.001 g or 0.05 g), preferably 0.005 g to 10 g, more preferably 0.005 g to 4 g, per kilogram of seed.

In another aspect the present invention provides an insecticidal, acaricidal, nematicidal or molluscicidal composition comprising an insecticidally, acaricidally, nematicidally or molluscicidally effective amount of a compound of formula (I) and a suitable carrier or diluent therefor. The composition is preferably an insecticidal, acaricidal, nematicidal or molluscicidal composition.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of formula (I).

Dustable powders (DP) may be prepared by mixing a compound of formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers) and mechanically grinding the mixture to a fine powder.

Soluble powders (SP) may be prepared by mixing a compound of formula (I) with one or more water-soluble inorganic salts (such as sodium bicarbonate, sodium carbonate or magnesium sulfate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula (I) and one or more powdered solid diluents or carriers, or from pre-formed blank granules by absorbing a compound of formula (I) (or a solution thereof, in a suitable agent) in a porous granular material (such as pumice, attapulgite clays, fuller's earth, kieselguhr, diatomaceous earths or ground corn cobs) or by adsorbing a compound of formula (I) (or a solution thereof, in a suitable agent) on to a hard core material (such as sands, silicates, mineral carbonates, sulfates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula (I) in water or an organic solvent, such as a ketone, alcohol or glycol ether. These solutions may contain a surface active agent (for example to improve water dilution or prevent crystallization in a spray tank).

Emulsifiable concentrates (EC) or oil-in-water emulsions (EW) may be prepared by dissolving a compound of formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula (I) either as a liquid (if it is not a liquid at room temperature, it may be melted at a reasonable temperature, typically below 70° C.) or in solution (by dissolving it in an appropriate solvent) and then emulsifiying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of formula (I) is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula (I). SCs may be prepared by ball or bead milling the solid compound of formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of formula (I) may be dry milled and added to water, containing agents hereinbefore described, to produce the desired end product.

Aerosol formulations comprise a compound of formula (I) and a suitable propellant (for example n-butane). A compound of formula (I) may also be dissolved or dispersed in a suitable medium (for example water or a water miscible liquid, such as n-propanol) to provide compositions for use in non-pressurized, hand-actuated spray pumps.

A compound of formula (I) may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerization stage such that an aqueous dispersion of oil droplets is obtained, in which each oil droplet is encapsulated by a polymeric shell and contains a compound of formula (I) and, optionally, a carrier or diluent therefor. The polymeric shell may be produced by either an interfacial polycondensation reaction or by a coacervation procedure. The compositions may provide for controlled release of the compound of formula (I) and they may be used for seed treatment. A compound of formula (I) may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of a compound of formula (I)). Such additives include surface active agents, spray additives based on oils, for example certain mineral oils or natural plant oils (such as soy bean and rape seed oil), and blends of these with other bio-enhancing adjuvants (ingredients which may aid or modify the action of a compound of formula (I)).

A compound of formula (I) may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

Wetting agents, dispersing agents and emulsifying agents may be surface SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulfuric acid (for example sodium lauryl sulfate), salts of sulfonated aromatic compounds (for example sodium dodecylbenzenesulfonate, calcium dodecylbenzenesulfonate, butylnaphthalene sulfonate and mixtures of sodium di-isopropyl- and tri-isopropyl-naphthalene sulfonates), ether sulfates, alcohol ether sulfates (for example sodium laureth-3-sulfate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulfosuccinamates, paraffin or olefine sulfonates, taurates and lignosulfonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of formula (I) may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapor or applied through distribution or incorporation of a composition (such as a granular composition or a composition packed in a water-soluble bag) in soil or an aqueous environment.

A compound of formula (I) may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs, SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula (I) (for example 0.0001 to 10%, by weight) depending upon the purpose for which they are to be used.

A compound of formula (I) may be used in mixtures with fertilizers (for example nitrogen-, potassium- or phosphorus-containing fertilizers). Suitable formulation types include granules of fertilizer. The mixtures preferably contain up to 25% by weight of the compound of formula (I).

The invention therefore also provides a fertilizer composition comprising a fertilizer and a compound of formula (I).

The compositions of this invention may contain other compounds having biological activity, for example micronutrients or compounds having fungicidal activity or which possess plant growth regulating, herbicidal, insecticidal, nematicidal or acaricidal activity.

The compound of formula (I) may be the sole active ingredient of the composition or it may be admixed with one or more additional active ingredients such as a pesticide, fungicide, synergist, herbicide or plant growth regulator where appropriate. An additional active ingredient may: provide a composition having a broader spectrum of activity or increased persistence at a locus; synergize the activity or complement the activity (for example by increasing the speed of effect or overcoming repellency) of the compound of formula (I); or help to overcome or prevent the development of resistance to individual components. The particular additional active ingredient will depend upon the intended utility of the composition. Examples of suitable pesticides include the following:

a) Pyrethroids, such as permethrin, cypermethrin, fenvalerate, esfenvalerate, deltamethrin, cyhalothrin (in particular lambda-cyhalothrin), bifenthrin, fenpropathrin, cyfluthrin, tefluthrin, fish safe pyrethroids (for example ethofenprox), natural pyrethrin, tetramethrin, S-bioallethrin, fenfluthrin, prallethrin or 5-benzyl-3-furylmethyl-(E)-(1R,3S)-2,2-dimethyl-3-(2-oxothiolan-3-ylidenemethyl)cyclopropane carboxylate;
b) Organophosphates, such as profenofos, sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate or diazinon;
c) Carbamates (including aryl carbamates), such as pirimicarb, triazamate, cloethocarb, carbofuran, furathiocarb, ethiofencarb, aldicarb, thiofurox, carbosulfan, bendiocarb, fenobucarb, propoxur, methomyl or oxamyl;
d) Benzoyl ureas, such as diflubenzuron, triflumuron, hexaflumuron, flufenoxuron or chlorfluazuron;
e) Organic tin compounds, such as cyhexatin, fenbutatin oxide or azocyclotin;
f) Pyrazoles, such as tebufenpyrad and fenpyroximate;
g) Macrolides, such as avermectins or milbemycins, for example abamectin, emamectin benzoate, ivermectin, milbemycin, spinosad, azadirachtin or spinetoram;
h) Hormones or pheromones;
i) Organochlorine compounds, such as endosulfan (in particular alpha-endosulfan), benzene hexachloride, DDT, chlordane or dieldrin;
j) Amidines, such as chlordimeform or amitraz;
k) Fumigant agents, such as chloropicrin, dichloropropane, methyl bromide or metam;
l) Neonicotinoid compounds, such as imidacloprid, thiacloprid, acetamiprid, nitenpyram, dinotefuran, thiamethoxam, clothianidin, nithiazine or flonicamid;
m) Diacylhydrazines, such as tebufenozide, chromafenozide or methoxyfenozide;
n) Diphenyl ethers, such as diofenolan or pyriproxifen;

### o) Indoxacarb;

### p) Chlorfenapyr;

### q) Pymetrozine;

r) Spirotetramat, spirodiclofen or spiromesifen;
s) Diamides, such as flubendiamide, chlorantraniliprole (Rynaxypyr®) or cyantraniliprole;

### t) Sulfoxaflor; or

### u) Metaflumizone.

In addition to the major chemical classes of pesticide listed above, other pesticides having particular targets may be employed in the composition, if appropriate for the intended utility of the composition. For instance, selective insecticides for particular crops, for example stemborer specific insecticides (such as cartap) or hopper specific insecticides (such as buprofezin) for use in rice may be employed. Alternatively insecticides or acaricides specific for particular insect species/stages may also be included in the compositions (for example acaricidal ovo-larvicides, such as clofentezine, flubenzimine, hexythiazox or tetradifon; acaricidal motilicides, such as dicofol or propargite; acaricides, such as bromopropylate or chlorobenzilate; or growth regulators, such as hydramethylnon, cyromazine, methoprene, chlorfluazuron or diflubenzuron).

Examples of fungicidal compounds which may be included in the composition of the invention are (E)-N-methyl-2-[2-(2,5-dimethylphenoxymethyl)phenyl]-2-methoxy-iminoacetamide (SSF-129), 4-bromo-2-cyano-N,N-dimethyl-6-trifluoromethyl-benzimidazole-1-sulfonamide, α-[N-(3-chloro-2,6-xylyl)-2-methoxyacetamido]-γ-butyrolactone, 4-chloro-2-cyano-N,N-dimethyl-5-p-tolylimidazole-1-sulfonamide (IKF-916, cyamidazosulfamid),

3-5-dichloro-N-(3-chloro-1-ethyl-1-methyl-2-oxopropyl)-4-methylbenzamide (RH-7281, zoxamide), N-allyl-4,5,-dimethyl-2-trimethylsilylthiophene-3-carboxamide (MON65500), N-(1-cyano-1,2-dimethylpropyl)-2-(2,4-dichlorophenoxy)propionamide (AC382042), N-(2-methoxy-5-pyridyl)-cyclopropane carboxamide, acibenzolar (CGA245704), alanycarb, aldimorph, anilazine, azaconazole, azoxystrobin, benalaxyl, benomyl, biloxazol, bitertanol, blasticidin S, bromuconazole, bupirimate, captafol, captan, carbendazim, carbendazim chlorhydrate, carboxin, carpropamid, carvone, CGA41396, CGA41397, chinomethionate, chlorothalonil, chlorozolinate, clozylacon, copper containing compounds such as copper oxychloride, copper oxyquinolate, copper sulfate, copper tallate and Bordeaux mixture, cymoxanil, cyproconazole, cyprodinil, debacarb, di-2-pyridyl disulfide 1,1′-dioxide, dichlofluanid, diclomezine, dicloran, diethofencarb, difenoconazole, difenzoquat, diflumetorim, O,O-di-iso-propyl-S-benzyl thiophosphate, dimefluazole, dimetconazole, dimethomorph, dimethirimol, diniconazole, dinocap, dithianon, dodecyl dimethyl ammonium chloride, dodemorph, dodine, doguadine, edifenphos, epoxiconazole, ethirimol, ethyl-(Z)-N-benzyl-N-([methyl(methyl-thioethylideneaminooxycarbonyl)amino]thio)-β-alaninate, etridiazole, famoxadone, fenamidone (RPA407213), fenarimol, fenbuconazole, fenfuram, fenhexamid (KBR2738), fenpiclonil, fenpropidin, fenpropimorph, fentin acetate, fentin hydroxide, ferbam, ferimzone, fluazinam, fludioxonil, flumetover, fluoroimide, fluquinconazole, flusilazole, flutolanil, flutriafol, folpet, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb (SZX0722), isopropanyl butyl carbamate, isoprothiolane, kasugamycin, kresoxim-methyl, LY186054, LY211795, LY248908, mancozeb, maneb, mefenoxam, mepanipyrim, mepronil, metalaxyl, metconazole, metiram, metiram-zinc, metominostrobin, myclobutanil, neoasozin, nickel dimethyldithiocarbamate, nitrothal-isopropyl, nuarimol, ofurace, organomercury compounds, oxadixyl, oxasulfuron, oxolinic acid, oxpoconazole, oxycarboxin, pefurazoate, penconazole, pencycuron, phenazin oxide, phosetyl-A1, phosphorus acids, phthalide, picoxystrobin (ZA1963), polyoxin D, polyram, probenazole, prochloraz, procymidone, propamocarb, propiconazole, propineb, propionic acid, pyrazophos, pyrifenox, pyrimethanil, pyroquilon, pyroxyfur, pyrroInitrin, quaternary ammonium compounds, quinomethionate, quinoxyfen, quintozene, sipconazole (F-155), sodium pentachlorophenate, spiroxamine, streptomycin, sulfur, tebuconazole, tecloftalam, tecnazene, tetraconazole, thiabendazole, thifluzamid, 2-(thiocyanomethylthio)benzothiazole, thiophanate-methyl, thiram, timibenconazole, tolclofos-methyl, tolylfluanid, triadimefon, triadimenol, triazbutil, triazoxide, tricyclazole, tridemorph, trifloxystrobin (CGA279202), triforine, triflumizole, triticonazole, validamycin A, vapam, vinclozolin, zineb and ziram.

The compounds of formula (I) may be mixed with soil, peat or other rooting media for the protection of plants against seed-borne, soil-borne or foliar fungal diseases.

Examples of suitable synergists for use in the compositions include piperonyl butoxide, sesamex, safroxan and dodecyl imidazole.

Suitable herbicides and plant-growth regulators for inclusion in the compositions will depend upon the intended target and the effect required.

An example of a rice selective herbicide which may be included is propanil. An example of a plant growth regulator for use in cotton is PIX™.

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

The following Examples illustrate, but do not limit, the invention.

### Preparation Examples

The following abbreviations were used in this section: s=singlet; bs=broad singlet; d=doublet; dd=double doublet; dt=double triplet; t=triplet, tt=triple triplet, q=quartet, sept=septet; m=multiplet; Me=methyl; Et=ethyl; Pr=propyl; Bu=butyl; M.p.=melting point; RT=retention time, [M+H]⁺=molecular mass of the molecular cation, [M−H]⁻=molecular mass of the molecular anion.

The following LC-MS methods were used to characterize the compounds:

### Method B

| MS | ZMD Mass Spectrometer from Waters (single quadrupole mass spectrometer), | | | |
|---|---|---|---|---|
| | ionization method: electrospray, polarity: positive ionization, capillary (kV) | | | |
| | 3.00, cone (V) 30.00, extractor (V) 3.00, source temperature (° C.) 150, | | | |
| | desolvation temperature (° C.) 320, cone gas flow (L/Hr) 50, desolvation gas | | | |
| | flow (L/Hr) 400, mass range: 150 to 800 Da. | | | |
| LC | Alliance 2795 LC HPLC from Waters: quaternary pump, heated column | | | |
| | compartment and diode-array detector. | | | |
| | Column: Waters Atlantis dc18, length (mm) 20, internal diameter (mm) 3, | | | |
| | particle size (μm) 3, temperature (° C.) 40, DAD wavelength range (nm): 200 to | | | |
| | 500, solvent gradient: A = 0.1% v/v formic acid in water and B = 0.1% v/v | | | |
| | formic acid in acetonitrile. | | | |
| | Time (min) | A % | B % | Flow (ml/min) |
| | 0.0 | 80 | 20 | 1.7 |
| | 5.0 | 0.0 | 100 | 1.7 |
| | 5.6 | 0.0 | 100 | 1.7 |
| | 6.0 | 80 | 20 | 1.7 |

### Method E

| MS | ZQ Mass Spectrometer from Waters (single quadrupole mass spectrometer), | | | |
|---|---|---|---|---|
| | ionization method: electrospray, polarity: positive ionization, capillary (kV) | | | |
| | 3.00, cone (V) 30.00, extractor (V) 3.00, source temperature (° C.) 100, | | | |
| | desolvation temperature (° C.) 200, cone gas flow (L/Hr) 200, desolvation gas | | | |
| | flow (L/Hr) 250, mass range: 150 to 800 Da. | | | |
| LC | 1100er Series HPLC from Agilent: quaternary pump, heated column | | | |
| | compartment and diode-array detector. | | | |
| | Column: Waters Atlantis dc18, length (mm) 20, internal diameter (mm) 3, | | | |
| | particle size (μm) 3, temperature (° C.) 40, DAD wavelength range (nm): 200 to | | | |
| | 500, solvent gradient: A = 0.1% v/v formic acid in water and B = 0.1% v/v | | | |
| | formic acid in acetonitrile. | | | |
| | Time (min) | A % | B % | Flow (ml/min) |
| | 0.0 | 80 | 20 | 1.7 |
| | 2.5 | 0.0 | 100 | 1.7 |
| | 2.8 | 0.0 | 100 | 1.7 |
| | 2.9 | 80 | 20 | 1.7 |

### EXAMPLE I1

### Preparation of 4-[5-(3,5-dichloro-phenyl)-4-(1-hydroxy-ethyl)-5-trifluoro-methyl-4,5-dihydro-isoxazol-3-yl]-2-methyl-benzoic acid methyl ester

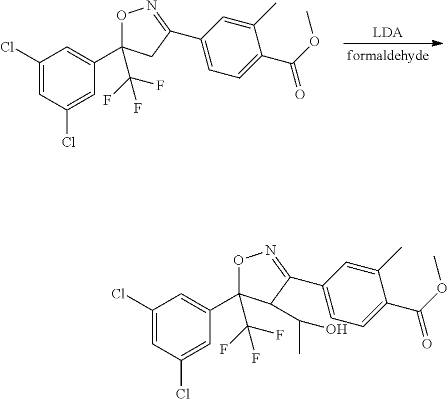 [SMILES: COC(=O)c1ccc(C2=NOC(c3cc(Cl)cc(Cl)c3)(C(F)(F)F)C2)cc1C ; COC(=O)c1ccc(C2=NOC(c3cc(Cl)cc(Cl)c3)(C(F)(F)F)C2C(C)O)cc1C]

To a solution of N,N-diisopropylamine (0.24 ml) in dry tetrahydrofuran (6 ml) stirred under argon at 0° C., was added butyl lithium (“BuLi”) (2.5 M in hexane) (0.80 ml). The solution was stirred at 0° C. for 30 minutes then was cooled to −85° C. To this solution was added a solution of 4-[5-(3,5-dichloro-phenyl)-5-trifluoromethyl-4,5-dihydro-isoxazol-3-yl]-2-methyl-benzoic acid methyl ester (made as described in EP 1,731,512) (404 mg) in dry tetrahydrofuran (3 ml). The reaction mixture was stirred at −85° C. until deprotonation was completed as monitored by thin layer chromatography. Then, to this solution was added acetaldehyde (0.14 ml) and the reaction mixture was stirred at −85° C. for 1.5 hours. The reaction was quenched by addition of aqueous ammonium chloride (saturated) at −85° C. The mixture allowed to warm to ambient temperature and was then extracted with dichloromethane. The combined organic extracts were dried over magnesium sulfate and concentrated. The residue was purified by chromatography on silica gel (eluent: heptane/diethyl ether 60:40) to give 4-[5-(3,5-dichloro-phenyl)-4-(1-hydroxy-ethyl)-5-trifluoro-methyl-4,5-dihydro-isoxazol-3-yl]-2-methyl-benzoic acid methyl ester (290 mg) as a white solid. ¹H-NMR (CDCl₃, 400 MHz): 7.98-7.96 (d, 1H), 7.60-7.45 (m, 5H), 4.09-4.08 (m, 1H), 3.96-3.91 (m, 1H), 3.91 (s, 3H), 2.63 (s, 3H), 1.07 and 0.94 (d, 3H).

Similarly, 4-[5-(3,5-Dichloro-phenyl)-4-(hydroxy-phenyl-methyl)-5-trifluoromethyl-4,5-dihydro-isoxazol-3-yl]-2-methyl-benzoic acid tert-butyl ester was obtained using benzaldehyde. ¹H-NMR (CDCl₃, 400 MHz): 7.68 (bs, 2H), 7.57 (d, 1H), 7.47 (m, 1H), 7.21-7.08 (m, 4H), 6.93 (d, 2H), 6.73 (s, 1H), 4.88 (m, 1H), 4.36 (m, 1H), 2.27 (s, 3H), 1.59 (s, 9H).

Similarly, 4-[5-(3,5-Dichloro-phenyl)-4-hydroxy-5-trifluoromethyl-4,5-dihydro-isoxazol-3-yl]-2-methyl-benzoic acid tert-butyl ester was obtained using 2-Benzenesulfonyl-3-phenyl-oxaziridine. ¹H-NMR (CDCl₃, 400 MHz): 7.73 (d, 1H), 7.68 (s, 1H), 7.62 (d, 1H), 7.57 (s, 2H), 7.47 (m, 1H), 5.78 (bs, 1H), 2.55 (s, 3H), 1.58 (s, 9H).

### EXAMPLE I2

### Preparation of 4-[5-(3,5-dichloro-phenyl)-4-ethylidene-5-trifluoromethyl-4,5-dihydro-isoxazol-3-yl]-2-methyl-benzoic acid

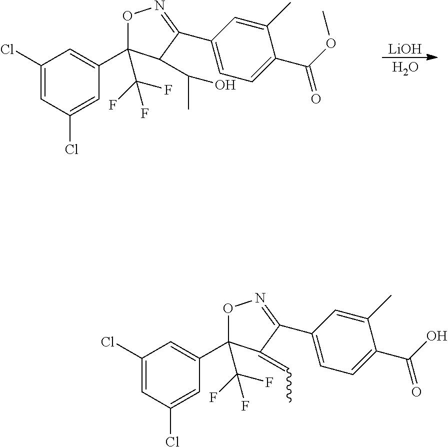

A mixture of 4-[5-(3,5-dichloro-phenyl)-4-(1-hydroxy-ethyl)-5-trifluoromethyl-4,5-dihydro-isoxazol-3-yl]-2-methyl-benzoic acid methyl ester (Example I1) (0.242 g, 0.51 mmol), lithium hydroxide monohydrate (0.06 g, 1.4 mmol), tetrahydrofuran (5 ml) and water (5 ml) was stirred at ambient temperature for 2 days. Then further portions of lithium hydroxide monohydrate were added to complete the reaction. In total, 545 mg of lithium hydroxide monohydrate were added over 5 days. The mixture was concentrated and the residue dissolved in water. The solution was acidified by addition of aqueous hydrochloric acid (1N) and extracted with ethyl acetate. The combined organic extracts were dried over magnesium sulfate and concentrated to give a residue which was used without further purification in the following step. LC/MS showed the presence of 4-[5-(3,5-dichloro-phenyl)-4-ethylidene-5-trifluoromethyl-4,5-dihydro-isoxazol-3-yl]-2-methyl-benzoic acid: RT=2.20 min, m/z=442/444/446 (M−H⁺).

### EXAMPLE I3

### Preparation of 4-{5-(3,5-Dichloro-phenyl)-4-[1-phenyl-meth-(E)-ylidene]-5-trifluoromethyl-4,5-dihydro-isoxazol-3-yl}-2-methyl-benzoic acid tert-butyl ester

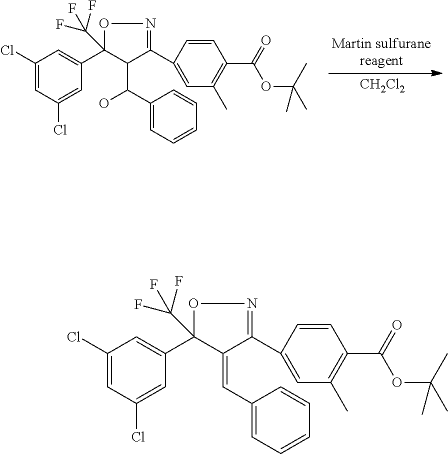

To a solution of 4-[5-(3,5-Dichloro-phenyl)-4-(hydroxy-phenyl-methyl)-5-trifluoromethyl-4,5-dihydro-isoxazol-3-yl]-2-methyl-benzoic acid tert-butyl ester (0.530 g, 0.91 mmol) in dichloromethane (15 mL) was added martin's sulfurane dehydrating agent ((Bis[α,α-bis(trifluoromethyl)benzyloxy]diphenylsulfur), (0.673 g, 1 mmol) and the solution was stirred at ambient temperature for 3 hours. Then a further portion of martin's sulfurane (0.01 g) was added to complete the reaction. The solution was stirred at ambient temperature for 19 hours. Then water was added and the solution was extracted with ethyl acetate. The combined organic extracts were dried over magnesium sulfate and concentrated to give a residue which was purified first by chromatography on silica gel (eluent: dichloromethane) then by cristallization to give 4-{5-(3,5-Dichloro-phenyl)-4-[1-phenyl-meth-(E)-ylidene]-5-trifluoromethyl-4,5-dihydro-isoxazol-3-yl}-2-methyl-benzoic acid tert-butyl ester as white crystals (442 mg). ¹H-NMR (CDCl₃, 400 MHz): 7.65 (m, 2H), 7.56 (d, 1H), 7.48 (m, 1H), 7.20 (m, 1H), 7.13 (t, 1H), 7.05 (d, 1H), 7.01 (t, 1H), 6.87 (d, 1H), 6.80 (m, 1H), 2.25 (s, 3H), 1.58 (s, 9H).

### EXAMPLE I4

### Preparation of 4-[3-(3,5-dichloro-phenyl)-4,4,4-trifluoromethyl-1-hydroxyimino-but-2-enyl]-2-methyl-benzoic acid tert-butyl ester

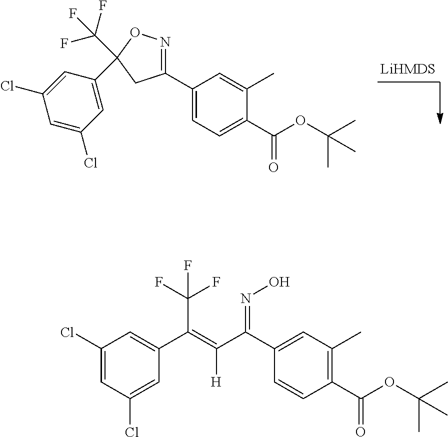 [SMILES: Cc1cc(C2=NOC(c3cc(Cl)cc(Cl)c3)(C(F)(F)F)C2)ccc1C(=O)OC(C)(C)C ; Cc1cc(C(/C=C(/c2cc(Cl)cc(Cl)c2)C(F)(F)F)=N\O)ccc1C(=O)OC(C)(C)C]

To a solution of 4-[5-(3,5-dichloro-phenyl)-5-trifluoromethyl-4,5-dihydro-isoxazol-3-yl]-2-methyl-benzoic acid tert-butyl ester (preparation of similar compounds described in, for example, EP 1,731,512) (5 g) in dry tetrahydrofuran (110 ml) under at atmosphere of argon was added lithium bis(trimethylsilyl)amide (“LiHMDS”) (1M in tetrahydrofuran) (11 ml) at ambient temperature. Then, more lithium bis(trimethylsilyl)amide (“LiHMDS”) (1M in tetrahydrofuran) (6 ml in total) was added portionwise to the reaction mixture until completion of the reaction was observed. Then, the reaction mixture was quenched by addition of aqueous ammonium chloride (saturated). The mixture was extracted several times with diethyl ether. The combined organic extracts were dried over magnesium sulfate and concentrated. The residue was purified by column chromatography on silica gel (eluent: heptane/diethyl ether 9:1) to give 4-[3-(3,5-dichloro-phenyl)-4,4,4-trifluoromethyl-1-hydroxyimino-but-2-enyl]-2-methyl-benzoic acid tert-butyl ester (2 g). ¹H-NMR (CDCl₃, 400 MHz): 7.87 (d, 1H), 7.69 (s, 1H), 7.49-7.43 (m, 4H), 6.77 (s, 1H), 2.61 (s, 1H), 1.61 8 s, 9H).

### EXAMPLE I5

### Preparation of 4-[5-(3,5-dichloro-phenyl)-4-hydroxyimino-5-trifluoromethyl-4,5-dihydro-isoxazol-3-yl]-2-methyl-benzoic acid tert-butyl ester

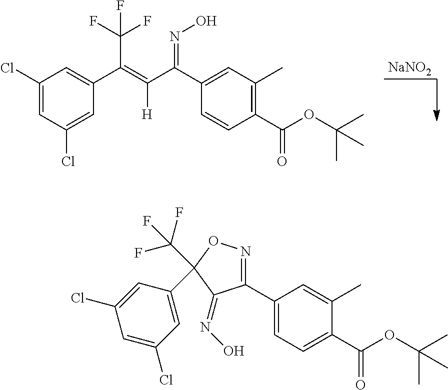 [SMILES: Cc1cc(C(/C=C(/c2cc(Cl)cc(Cl)c2)C(F)(F)F)=N\O)ccc1C(=O)OC(C)(C)C ; "NaNO2" ; Cc1cc(C2=NOC(c3cc(Cl)cc(Cl)c3)(C(F)(F)F)/C2=N/O)ccc1C(=O)OC(C)(C)C]

To a solution of 4-[3-(3,5-dichloro-phenyl)-4,4,4-trifluoromethyl-1-hydroxyimino-but-2-enyl]-2-methyl-benzoic acid tert-butyl ester (Example 13.1) (1 g) in a mixture of ethanol/water/tetrahydrofuran (1:2:2) (25 ml) was added sodium nitrite (500 mg). The reaction mixture was acidified to pH 1.5 by addition of aqueous hydrochloric acid (2M). The reaction mixture was stirred at ambient temperature for 24 hours. Further sodium nitrite (400 mg) was added and the reaction mixture acidified to pH 1.5 by addition of further aqueous hydrochloric acid (2M) and the reaction mixture was stirred at ambient temperature for 2 hours. Further sodium nitrite (100 mg) was added and the reaction mixture acidified to pH 1.5 by addition of further aqueous hydrochloric acid (2M) and the reaction mixture was stirred at ambient temperature for 24 hours. Further sodium nitrite (200 mg) was added and the reaction mixture acidified to pH 1.5 by addition of further aqueous hydrochloric acid (2M) and the reaction mixture was stored at ambient temperature for 48 hours. The reaction mixture was diluted with dichloromethane and water. The phases were separated and the organic layer was washed successively with water and brine, dried over magnesium sulfate and concentrated. The residue was purified by column chromatography on silica gel (eluent: heptane/diethyl ether 9:1) to give 4-{5-(3,5-dichloro-phenyl)-4-hydroxyimino-5-trifluoromethyl-4,5-dihydro-isoxazol-3-yl}-2-methyl-benzoic acid tert-butyl ester (512 mg) as yellow solid.

### EXAMPLE I6

### Preparation of 4-[5-(3,5-Dichloro-phenyl)-4-oxo-5-trifluoromethyl-4,5-dihydro-isoxazol-3-yl]-2-methyl-benzoic acid tert-butyl ester

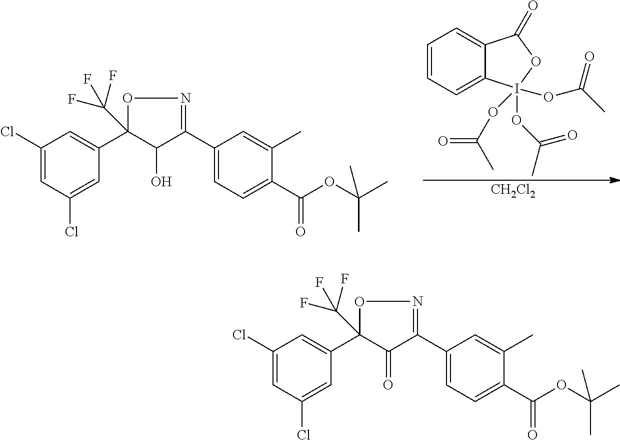

To a solution of 4-[5-(3,5-Dichloro-phenyl)-4-hydroxy-5-trifluoromethyl-4,5-dihydro-isoxazol-3-yl]-2-methyl-benzoic acid tert-butyl ester (0.900 g, 1.83 mmol) in dichloromethane (15 mL) was added Dess-martin's reagent (1,1,1-Tris(acetyloxy)-1,1-dihydro-1,2-benziodoxol-3-(1H)-one), (1.2 g, 2.75 mmol) and the solution was stirred at ambient temperature for 24 hours. Then a further portion of Dess-martin's reagent (0.40 g) was added to complete the reaction. The solution was stirred at ambient temperature for 3 hours. Then water was added and the solution was extracted with dichloromethane. The combined organic extracts were dried over magnesium sulfate and concentrated to give a residue which was purified first by chromatography on silica gel (eluent: heptanes/dichloromethane, ratio 3:2) to give 4-[5-(3,5-Dichloro-phenyl)-4-oxo-5-trifluoromethyl-4,5-dihydro-isoxazol-3-yl]-2-methyl-benzoic acid tert-butyl ester as a yellow oil (288 mg). ¹H-NMR (d6 DMSO, 400 MHz): 7.92 (m, 1H), 7.88 and 7.87 (m, 3H), 7.72 (m, 2H), 2.54 (s, 3H), 1.56 8 s, 9H).

### EXAMPLE I7

### Preparation of 4-[5-(3,5-dichloro-phenyl)-4-hydroxyimino-5-trifluoromethyl-4,5-dihydro-isoxazol-3-yl]-2-methyl-benzoic acid

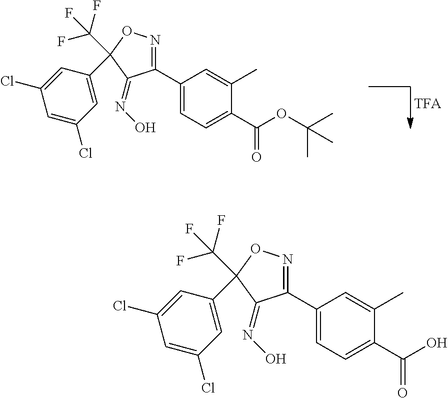 [SMILES: Cc1cc(C2=NOC(c3cc(Cl)cc(Cl)c3)(C(F)(F)F)/C2=N/O)ccc1C(=O)OC(C)(C)C ; Cc1cc(C2=NOC(c3cc(Cl)cc(Cl)c3)(C(F)(F)F)/C2=N/O)ccc1C(=O)O]

To a solution of 4-[5-(3,5-dichloro-phenyl)-4-hydroxyimino-5-trifluoromethyl-4,5-dihydro-isoxazol-3-yl]-2-methyl-benzoic acid tert-butyl ester (Example I3.2) (634 mg) in dichloromethane (4 ml) was added trifluoroacetic acid (“TFA”) (0.2 ml) and the reaction mixture was stirred at ambient temperature for 8 hours. Further trifluoroacetic acid (0.2 ml) was added and the reaction mixture was stirred at ambient temperature for 16 hours. The reaction mixture was diluted with ethyl acetate and water and the phases were separated. The organic layer was washed successively with water and brine, dried over magnesium sulfate, and concentrated to give 4-[5-(3,5-dichloro-phenyl)-4-hydroxyimino-5-trifluoromethyl-4,5-dihydro-isoxazol-3-yl]-2-methyl-benzoic acid (554 mg) as a yellow foam. ¹H-NMR (CDCl₃, 400 MHz): 8.94 (s, 1H), 8.10 (d, 1H), 7.90 (m, 2H), 7.59 (m, 2H), 7.44 (m, 1H), 2.69 (s, 3H).

Similarly, 4-{5-(3,5-Dichloro-phenyl)-4-[1-phenyl-methylidene]-5-trifluoromethyl-4,5-dihydro-isoxazol-3-yl}-2-methyl-benzoic acid was obtained. ¹H-NMR (CDCl₃, 400 MHz): 7.79 (d, 1H), 7.64 (m, 2H), 7.49 (m, 1H), 7.23 (bs, 1H), 7.10 (m, 2H), 7.00 (t, 1H), 6.89 (t, 1H), 2.36 (s, 3H).

### EXAMPLE P1

### Preparation of N-butyl-4-[5-(3,5-dichloro-phenyl)-4-ethylidene-5-trifluoro-methyl-4,5-dihydro-isoxazol-3-yl]-2-methyl-benzamide (Compound No. A1 of Table A)

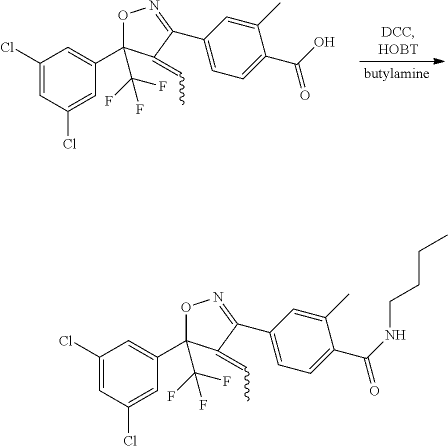

To a solution of 4-[5-(3,5-dichloro-phenyl)-4-ethylidene-5-trifluoromethyl-4,5-dihydro-isoxazol-3-yl]-2-methyl-benzoic acid (Example I2) (0.070 g) and butylamine (0.02 ml, 0.21 mmol) in dichloromethane (2 ml) under argon was added 1-hydroxybenzotriazole (“HOBT”) (21 mg, 0.15 mmol) then N,N′-dicyclohexylcarbodiimide (“DCC”) (35 mg, 0.17 mmol). The solution was stirred at ambient temperature for 45 minutes then allowed to stand at ambient temperature over 4 days. The solvent was evaporated and the residue was purified by column chromatography on silica gel (eluent: heptane/ethyl acetate 1:0 to 1:1) to give Compound No. A1 of Table A (40 mg) as a white foam. ¹H-NMR (CDCl₃, 400 MHz): 7.51-7.36 (m, 6H), 6.40-6.34 (q, 1H), 5.73 (bs, 1H), 3.49-3.43 (t, 2H), 2.50 (s, 3H), 1.68-1.24 (m, 7H), 0.97 (t, 3H).

Similarly, 4-[5-(3,5-dichloro-phenyl)-4-ethylidene-5-trifluoromethyl-4,5-dihydro-isoxazol-3-yl]-2-methyl-N-thietan-3-yl-benzamide (Compound No. A2 of Table A) was obtained using thietan-3-ylamine. ¹H-NMR (CDCl₃, 400 MHz): 7.50-7.38 (m, 6H), 6.39-6.34 (q, 1H), 6.25 (bd, 1H), 5.46-5.39 (m, 1H), 3.51-3.37 (m, 4H), 2.49 (s, 3H), 1.67 and 1.60 (d, 3H).

### EXAMPLE P2

### Method for Preparing the Compounds of the Invention from a Carboxylic Acid

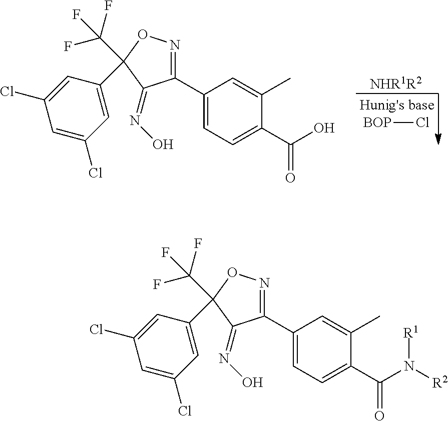

To a solution of the appropriate carboxylic acid (30 μmol), for example, 4-[5-(3,5-dichloro-phenyl)-4-hydroxyimino-5-trifluoromethyl-4,5-dihydro-isoxazol-3-yl]-2-methyl-benzoic acid (Example 1.5) for Compound No. B1 of Table B, in dimethylacetamide (0.4 ml) was added a solution of the appropriate amine (30 μmol), for example 1,1-dioxo-thietan-3-ylamine (preparation described in, for example, WO 2007/080131) for Compound No. B1 of Table B, in dimethylacetamide (0.145 ml) followed by diisopropylethylamine (Hunig's Base) (0.02 ml, 100 μmol) and a solution of bis(2-oxo-3-oxazolidinyl)phosphonic chloride (“BOP—Cl”) (15.3 mg) in dimethylacetamide (0.2 ml). The reaction mixture was stirred at 80° C. for 16 hours. Then the mixture was diluted with acetonitrile (0.6 ml) and a sample was used for the LC-MS analysis. The remaining mixture was further diluted with acetonitrile/dimethylformamide (4:1) (0.8 ml) and purified by HPLC to give the desired compound.

This method was used to make Compound Nos. B1, B2, B3 and B4 of Table B.

### EXAMPLE P3

### Preparation of 4-[5-(3,5-Dichloro-phenyl)-4-oxo-5-trifluoromethyl-4,5-dihydro-isoxazol-3-yl]-2-methyl-N-thietan-3-yl-benzamide (Compound No. D1 of Table D)

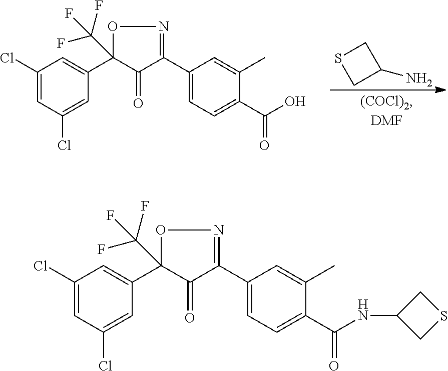 [SMILES: Cc1cc(C2=NOC(c3cc(Cl)cc(Cl)c3)(C(F)(F)F)C2=O)ccc1C(=O)O ; NC1CSC1 ; Cc1cc(C2=NOC(c3cc(Cl)cc(Cl)c3)(C(F)(F)F)C2=O)ccc1C(=O)NC1CSC1]

To a solution of 4-[5-(3,5-Dichloro-phenyl)-4-oxo-5-trifluoromethyl-4,5-dihydro-isoxazol-3-yl]-2-methyl-benzoic acid (0.048 g) in dichloromethane (1 ml) was added oxalyl chloride (0.01 ml). After addition of N,N-dimethylformamide (“DMF”) (2 drops) the reaction mixture was stirred at ambient temperature for 3 hours. Then the solution was evaporated under vacuo and the residue was dissolved in anhydrous dichloromethane (0.5 mL). This solution was stirred at ambient temperature for 22 hours. The reaction mixture was diluted with water and ethyl acetate and the phases were separated. The organic phase was washed twice with water, dried over sodium sulfate and concentrated. The residue was purified by chromatography on silica gel (eluent: dichloromethane/ethyl acetate 20:1) to give Compound No. D1 of Table D (28 mg) as a yellow crystals. ¹H-NMR (CDCl₃, 400 MHz): 7.95 (m, 2H), 7.75 (s, 2H), 7.51 (t, 1H), 7.47 (d, 1H), 6.22 (d, 1H), 5.46-5.40 (m, 1H), 3.52-3.48 and 3.42-3.38 (m, 4H), 2.50 (s, 3H).

### EXAMPLE P5

### Method for the preparation of 4-{5-(3,5-Dichloro-phenyl)-4-[methoxyimino]-5-trifluoromethyl-4,5-dihydro-isoxazol-3-yl}-2-methyl-N-thietan-3-yl-benzamide

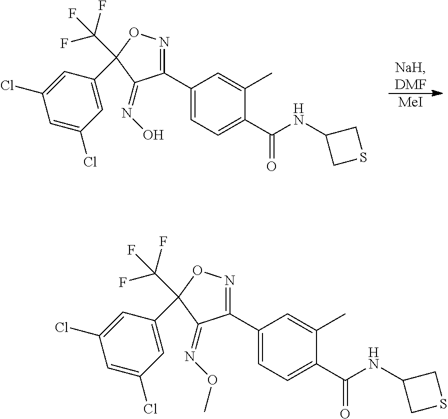

To a solution of 4-{5-(3,5-Dichloro-phenyl)-4-[hydroxyimino]-5-trifluoromethyl-4,5-dihydro-isoxazol-3-yl}-2-methyl-N-thietan-3-yl-benzamide (0.050 mg, 0.09 mmol) in dimethylformamide (3 mL) under argon, was added sodium hydride (60% suspension in mineral oil, 0.006 mg) followed by iodomethane (0.02 mg) and the solution was stirred at ambient temperature for 30 minutes. Then the mixture was diluted with saturated ammonium chloride and the solution was extracted with ethyl acetate. The combined organic extracts were dried over magnesium sulfate and concentrated to give a residue which was purified by chromatography on silica gel (eluent: heptane/ethyl acetate 1:0 to 4:1) to give 4-{5-(3,5-Dichloro-phenyl)-4-[methoxyimino]-5-trifluoromethyl-4,5-dihydro-isoxazol-3-yl}-2-methyl-N-thietan-3-yl-benzamide as a yellow oil (0.05 mg). ¹H-NMR (CDCl₃, 400 MHz): 7.90-7.87 (m, 2H), 7.53 (m, 2H), 7.46-7.45 (m, 2H), 6.24 (d, 1H), 5.46-5.42 (m, 1H), 4.12 (s, 3H), 3.53-3.48 and 3.42-3.38 (m, 4H), 2.50 (s, 3H).

Similarly, 4-{5-(3,5-Dichloro-phenyl)-4-[2,2-difluoro-ethoxyimino]-5-trifluoromethyl-4,5-dihydro-isoxazol-3-yl}-2-methyl-N-thietan-3-yl-benzamide was obtained using Trifluoro-methanesulfonic acid 2,2-difluoro-ethyl ester as an alkylating agent. ¹H-NMR (CDCl₃, 400 MHz): 7.85-7.78 (m, 2H), 7.52 (s, 2H), 7.47-7.44 (m, 2H), 6.06-5.78 (m, 1H), 5.47-5.41 (m, 1H), 4.53-4.45 (m, 2H), 3.51-3.40 (m, 4H), 2.49 (s, 3H).

TABLE A
Table A provides compounds of formula (Ia′) wherein G¹ is oxygen,
R³ is trifluoromethyl, R⁴ is 3,5-dichloro-phenyl-,
R⁵ is methyl, R^{6a} is hydrogen, R^{6b} is methyl, and R¹ and R² have the
values listed in the table below.

| | |
|---|---|
| | (Ia′) |
| 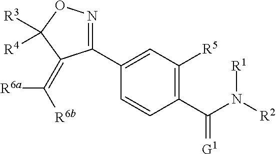 | |

| Compound | | | | | LC-MS |
|---|---|---|---|---|---|
| No. | R¹ | R² | RT (min) | [M + H]⁺ | method |
| A1 | H | butyl- | 2.28 | 497/ 499/ | E |
| | | | | 501 | |
| A2 | H | thietan-3-yl- | 2.18 | 559/ 561/ | E |
| | | | | 563 | |
| | | | | (+HCOOH) | |

TABLE B
Table B provides compounds of formula (Ic′) wherein G¹ is oxygen,
R³ is trifluoromethyl, R⁴ is 3,5-dichloro-phenyl-,
R⁵ is methyl, R⁷ is hydroxy, and R¹ and R² have the values listed in
the table below.

| | |
|---|---|
| | (Ic′) |
| 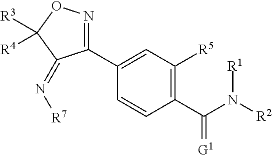 | |

| Compound | | | | | LC-MS |
|---|---|---|---|---|---|
| No. | R¹ | R² | RT (min) | [M + H]⁺ | method |
| B1 | H | 1,1-dioxo- | 2.6 | 591 | B |
| | | thietan-3-yl- | | | |
| B2 | H | 3-methyl- | 3.11 | 532 | B |
| | | thietan-3-yl- | | | |
| B3 | H | 1-oxo- | 2.35 | 534 | B |
| | | thietan-3-yl- | | | |
| B4 | H | thietan-3-yl- | 2.94 | 518 | B |

TABLE C
Table C provides compounds of formula (Ia′) wherein G¹ is oxygen,
R³ is trifluoromethyl, R⁴ is 3,5-dichloro-phenyl-,
R⁵ is methyl, R^{6a} is hydrogen, R^{6b} is phenyl, and R¹ and R² have the
values listed in the table below.

| | |
|---|---|
| | (Ia′) |
| 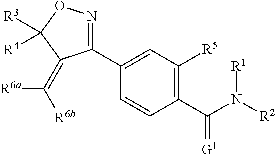 | |

| Compound | | | | | LC-MS |
|---|---|---|---|---|---|
| No. | R¹ | R² | RT (min) | [M + H]⁺ | method |
| C1 | H | butyl- | 3.67 | 561 | B |
| C2 | H | 2,2,2-trifluoro- | 3.57 | 586.9 | B |
| | | ethyl- | | | |
| C3 | H | ethyl- | 3.34 | 533.0 | B |
| C4 | H | 1-methoxy-prop-2- | 3.39 | 577.1 | B |
| | | yl- | | | |
| C5 | H | (1H-benzimidazol- | 2.4 | 635.0 | B |
| | | 2-yl)-methyl- | | | |
| C6 | H | 3,3,3-trifluoro- | 3.56 | 600.96 | B |
| | | propyl- | | | |
| C7 | H | 2-butyl- | 3.63 | 561 | B |
| C8 | H | (furan-2-yl)- | 3.34 | 589.03 | B |
| | | methyl- | | | |
| C9 | H | phenyl-methyl- | 3.68 | 594.98 | B |
| C10 | H | (2-fluoro-phenyl)- | 3.73 | 612.99 | B |
| | | methyl- | | | |
| C11 | H | (4-methoxy- | 3.67 | 624.95 | B |
| | | phenyl)-methyl- | | | |
| C12 | H | 1,1-dioxo-thietan- | 3.19 | 608.89 | B |
| | | 3-yl- | | | |
| C13 | H | (2-chloro-pyrid-5- | 3.49 | 631.93 | B |
| | | yl)-methyl- | | | |
| C14 | H | 3-fluoro-phenyl- | 3.94 | 598.9 | B |
| C15 | H | (pyrid-2-yl)- | 2.87 | 596.05 | B |
| | | methyl- | | | |
| C16 | H | 1,3-dimethyl-1H- | 3.29 | 599.08 | B |
| | | pyrazol-5-yl- | | | |
| C17 | H | 4-methyl-thiazol-2- | 3.76 | 601.96 | B |
| | | yl- | | | |
| C18 | H | 3-methyl-thietan-3- | 3.73 | 590.96 | B |
| | | yl- | | | |
| C19 | H | 1-oxo-thietan-3-yl- | 2.87 | 592.99 | B |
| C20 | H | thietan-3-yl- | 3.52 | 576.93 | B |
| C21 | H | cyclobutyl- | 3.58 | 559.04 | B |

TABLE D
Table D provides compounds of formula (Ic′) wherein G¹ is oxygen,
R³ is trifluoromethyl, R⁴ is 3,5-dichloro-phenyl-,
R⁵ is methyl, R⁷ is methoxy, and R¹ and R² have the values listed in
the table below.

| | |
|---|---|
| | (Ic′) |
| 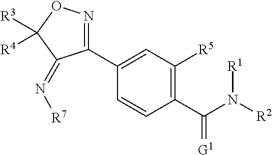 | |

| Compound | | | | | LC-MS |
|---|---|---|---|---|---|
| No. | R¹ | R² | RT (min) | [M − H]⁺ | method |
| D1 | H | thietan-3-yl- | 2.23 | 530/532 | E |

TABLE E
Table E provides compounds of formula (Ic′) wherein G¹ is oxygen,
R³ is trifluoromethyl, R⁴ is 3,5-dichloro-phenyl-,
R⁵ is methyl, R⁷ is 2,2 difluoroethoxy, and R¹ and R² have the values
listed in the table below.

| | |
|---|---|
| | (Ic′) |
| 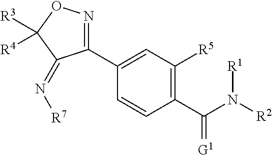 | |

| Compound | | | | | LC-MS |
|---|---|---|---|---|---|
| No. | R¹ | R² | RT (min) | [M + H]⁺ | method |
| E1 | H | thietan-3-yl- | 2.21 | 580/582 | B |

TABLE F
Table F provides compounds of formula (Ib′) wherein G¹ is oxygen,
R³ is trifluoromethyl, R⁴ is 3,5-dichloro-phenyl-,
R⁵ is methyl and R¹ and R² have the values listed in the table below.

| | |
|---|---|
| | (Ib′) |
| 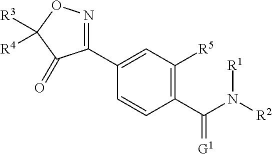 | |

| Compound | | | | | LC-MS |
|---|---|---|---|---|---|
| No. | R¹ | R² | RT (min) | [M − H]⁺ | method |
| F1 | H | thietan-3-yl- | 2.23 | 501/503 | B |

### Biological Examples

This Example illustrates the pesticidal/insecticidal properties of compounds of formula (I). Tests were performed as follows:

Spodoptera littoralis (Egyptian Cotton Leafworm):

Cotton leaf discs were placed on agar in a 24-well microtiter plate and sprayed with test solutions at an application rate of 200 ppm. After drying, the leaf discs were infested with 5 L1 larvae. The samples were checked for mortality, feeding behavior, and growth regulation 3 days after treatment (DAT).

The following compound gave at least 80% control of Spodoptera littoralis: A1, A2, B1.

Heliothis virescens (Tobacco Budworm):

Eggs (0-24 h old) were placed in 24-well microtiter plate on artificial diet and treated with test solutions at an application rate of 200 ppm (concentration in well 18 ppm) by pipetting. After an incubation period of 4 days, samples were checked for egg mortality, larval mortality, and growth regulation.

The following compound gave at least 80% control of Heliothis virescens: A1, A2, B1, B3, B4.

Plutella xylostella (Diamond Back Moth):

24-well microtiter plate (MTP) with artificial diet was treated with test solutions at an application rate of 200 ppm (concentration in well 18 ppm) by pipetting. After drying, the MTP's were infested with L2 larvae (7-12 per well). After an incubation period of 6 days, samples were checked for larval mortality and growth regulation.

The following compound gave at least 80% control of Plutella xylostella: A1, A2, B1, B3, B4, C15.

Diabrotica balteata (Corn Root Worm):

A 24-well microtiter plate (MTP) with artificial diet was treated with test solutions at an application rate of 200 ppm (concentration in well 18 ppm) by pipetting. After drying, the MTP's were infested with L2 larvae (6-10 per well). After an incubation period of 5 days, samples were checked for larval mortality and growth regulation.

The following compound gave at least 80% control of Diabrotica balteata: A¹, A², C19.

Thrips tabaci (Onion Thrips):

Sunflower leaf discs were placed on agar in a 24-well microtiter plate and sprayed with test solutions at an application rate of 200 ppm. After drying, the leaf discs were infested with an aphid population of mixed ages. After an incubation period of 7 days, samples were checked for mortality.

The following compounds gave at least 80% control of Thrips tabaci: A2, B1, B3, B4.

Tetranychus urticae (Two-Spotted Spider Mite):

Bean leaf discs on agar in 24-well microtiter plates were sprayed with test solutions at an application rate of 200 ppm. After drying, the leaf discs are infested with mite populations of mixed ages. 8 days later, discs are checked for egg mortality, larval mortality, and adult mortality.

The following compound gave at least 80% control of Tetranychus urticae: A², B1, B3, F1.

Compound No. B2 of Table B was tested using the same protocols and showed little or no effect on mortality, feeding behavior, or growth regulation under the test conditions.

1. A compound of formula (I) 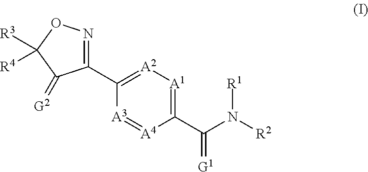
wherein
A¹, A², A³ and A⁴ are independently of each other C—H, C—R⁵, or nitrogen;
G¹ is oxygen or sulfur;
G² is C(R^{6a})(R^{6b}), oxygen, sulfur, or N—R⁷;
R¹ is hydrogen, C₁-C₈alkyl, C₁-C₈alkoxy-, C₁-C₈alkylcarbonyl-, or C₁-C₈alkoxycarbonyl-;
R² is C₁-C₈alkyl or C₁-C₈alkyl substituted by one to five R⁸, C₃-C₁₀cycloalkyl or C₃-C₁₀cycloalkyl substituted by one to five R⁹, aryl-C₁-C₄alkylene- or aryl-C₁-C₄alkylene- wherein the aryl moiety is substituted by one to five R¹⁰, heterocyclyl-C₁-C₄alkylene- or heterocyclyl-C₁-C₄alkylene- wherein the heterocyclyl moiety is substituted by one to five R¹⁰, aryl or aryl substituted by one to five R¹⁰, heterocyclyl or heterocyclyl substituted by one to five R¹⁰, C₁-C₈alkylaminocarbonyl-C₁-C₄ alkylene, C₁-C₈haloalkylaminocarbonyl-C₁-C₄ alkylene, or C₃-C₈cycloalkyl-aminocarbonyl-C₁-C₄ alkylene;
R³ is C₁-C₈haloalkyl;
R⁴ is aryl or aryl substituted by one to five R¹¹, or heteroaryl or heteroaryl substituted by one to five R¹¹;
each R⁵ is independently halogen, cyano, nitro, C₁-C₈alkyl, C₁-C₈haloalkyl, C₃-C₁₀cycloalkyl, C₁-C₈alkoxy-, C₁-C₈haloalkoxy-, C₁-C₈alkylthio-, C₁-C₈haloalkylthio-, C₁-C₈alkylsulfinyl-, C₁-C₈haloalkylsulfinyl-, C₁-C₈alkylsulfonyl-, or C₁-C₈haloalkylsulfonyl-;
R^{6a} and R^{6b} are independently of each other hydrogen, halogen, C₁-C₈alkyl or C₁-C₈alkyl substituted by one to five R¹², C₂-C₈alkenyl or C₂-C₈alkenyl substituted by one to five R¹², C₂-C₈alkynyl, C₂-C₈haloalkynyl, C₁-C₈alkoxy-, C₁-C₈haloalkoxy-, C₁-C₈alkylcarbonyl-, C₁-C₈alkoxycarbonyl-, aryl or aryl substituted by one to five R¹³, or heterocyclyl or heterocyclyl substituted by one to five R¹³, or NR¹⁴R¹⁵, wherein
R¹⁴ and R¹⁵ are independently hydrogen, C₁-C₈alkyl, C₁-C₈haloalkyl, C₁-C₈alkylcarbonyl-, C₁-C₈alkoxycarbonyl-, or
R¹⁴ and R¹⁵ together with the nitrogen atom to which they are attached form a 3 to 7 membered heterocyclic ring; or
R^{6a} and R^{6b} together with the carbon atom to which they are attached form a 3 to 7 membered carbocyclic or heterocyclic ring;
R⁷ is hydrogen, hydroxy, C₁-C₈alkyl or C₁-C₈alkyl substituted by one to five R¹⁶, C₃-C₁₀cycloalkyl, C₁-C₈alkoxy- or C₁-C₈alkoxy- substituted by one to five R¹⁶, (C₁-C₈alkyl)amino-, di(C₁-C₈alkyl)amino-, (C₁-C₈alkylcarbonyl)amino-, or (C₁-C₈alkoxycarbonyl)amino-;
each R⁸, R¹² and R¹⁶ is independently halogen, cyano, nitro, hydroxy, C₁-C₈alkoxy-, C₁-C₈haloalkoxy-, C₁-C₈alkylcarbonyl-, C₁-C₈alkoxycarbonyl-, mercapto, C₁-C₈alkylthio-, C₁-C₈haloalkylthio-, C₁-C₈alkylsulfinyl-, C₁-C₈haloalkylsulfinyl-, C₁-C₈alkylsulfonyl-, or C₁-C₈haloalkylsulfonyl-;
each R⁹ is independently halogen or C₁-C₈alkyl;
each R¹⁰, R¹¹ and R¹³ is independently halogen, cyano, nitro, C₁-C₈alkyl, C₁-C₈haloalkyl, C₂-C₈alkenyl, C₂-C₈haloalkenyl, C₂-C₈alkynyl, C₂-C₈haloalkynyl, hydroxy, C₁-C₈alkoxy-, C₁-C₈haloalkoxy-, mercapto, C₁-C₈alkylthio-, C₁-C₈haloalkylthio-, C₁-C₈alkylsulfinyl-, C₁-C₈haloalkylsulfinyl-, C₁-C₈alkylsulfonyl-, C₁-C₈haloalkylsulfonyl-, C₁-C₈alkylcarbonyl-, C₁-C₈alkoxycarbonyl-, aryl or aryl substituted by one to five R¹⁷, or heterocyclyl or heterocyclyl substituted by one to five R¹⁷;
each R¹⁷ is independently halogen, cyano, nitro, C₁-C₄alkyl, C₁-C₄haloalkyl, C₁-C₄alkoxy-, or C₁-C₄haloalkoxy-; or a salt or N-oxide thereof.

2. A compound according to claim 1 wherein A¹ is C—R⁵, A² is C—H, A³ is C—H or nitrogen and A⁴ is C—H or nitrogen.

3. A compound according to any claim 1 wherein G¹ is oxygen.

4. A compound according to claim 1 wherein G² is C(R^{6a})(R^{6b}) oxygen or N—R⁷.

5. A compound according to claim 1 wherein R¹ is hydrogen, methyl, ethyl, methylcarbonyl-, or methoxycarbonyl-.

6. A compound according to claim 1 wherein R² is C₁-C₈alkyl or C₁-C₈alkyl substituted by one to five R⁸, C₃-C₁₀cycloalkyl or C₃-C₁₀cycloalkyl substituted by one to five R⁹, phenyl-C₁-C₄alkylene- or phenyl-C₁-C₄alkylene- wherein the phenyl moiety is substituted by one to five R¹⁰, pyridyl-C₁-C₄alkylene- or pyridyl-C₁-C₄alkylene- wherein the pyridyl moiety is substituted by one to four R¹⁰, oxetanyl or oxetanyl substituted by one to five R¹⁰, thietanyl or thietanyl substituted by one to five R¹⁰, oxo-thietanyl or oxo-thietanyl substituted by one to five R¹⁰, or dioxo-thietanyl or dioxo-thietanyl substituted by one to five R¹⁰.

7. A compound according to claim 1 wherein R³ is chlorodifluoromethyl or trifluoromethyl.

8. A compound according to claim 1 wherein R⁴ is aryl or aryl substituted by one to five R¹¹.

9. A compound according to claim 1 wherein
A¹ is C—R⁵, A² is C—H, A³ is C—H and A⁴ is C—H;
G¹ is oxygen;
G² is C(R^{6a})(R^{6b}), oxygen, or N—R⁷;
R¹ is hydrogen, methyl or ethyl;
R² is C₁-C₈alkyl or C₁-C₈alkyl substituted by one to five R⁸, C₃-C₁₀cycloalkyl or C₃-C₁₀cycloalkyl substituted by one to five R⁹, phenyl-C₁-C₄alkylene- or phenyl-C₁-C₄alkylene- wherein the phenyl moiety is substituted by one to five R¹⁰, pyridyl-C₁-C₄alkylene- or pyridyl-C₁-C₄alkylene- wherein the pyridyl moiety is substituted by one to four R¹⁰, oxetanyl or oxetanyl substituted by one to five R¹⁰, thietanyl or thietanyl substituted by one to five R¹⁰, oxo-thietanyl or oxo-thietanyl substituted by one to five R¹⁰, dioxo-thietanyl or dioxo-thietanyl substituted by one to five R¹⁰, C₁-C₈alkylaminocarbonyl-C₁-C₄ alkylene, C₁-C₈haloalkylaminocarbonyl-C₁-C₄ alkylene, or C₃-C₈cycloalkyl-aminocarbonyl-C₁-C₄ alkylene;
R³ is chlorodifluoromethyl or trifluoromethyl;
R⁴ is 3,5-dibromo-phenyl-, 3,5-dichloro-phenyl-, 3,5-bis-(trifluoromethyl)-phenyl-, 3,4-dichloro-phenyl- or 3,4,5-trichloro-phenyl-;
R⁵ is bromo, chloro, fluoro, cyano, nitro, methyl, ethyl, trifluoromethyl, methoxy, difluoromethoxy, or trifluoromethoxy;
R^{6a} is hydrogen or C₁-C₆alkyl;
R^{6b} is C₁-C₆alkyl or phenyl;
R⁷ hydroxy or C₁-C₆alkoxy-;
each R⁸ is independently chloro, fluoro, or methoxy;
each R⁹ is methyl;
each R¹⁰ is independently bromo, chloro, fluoro, cyano, nitro, methyl, ethyl, trifluoromethyl, methoxy, difluoromethoxy, or trifluoromethoxy.

10. A compound according to claim 1 wherein
A¹ is C—R⁵, A² is C—H, A³ is C—H and A⁴ is C—H;
G¹ is oxygen;
G² is C(R^{6a})(R^{6b}), oxygen, or N—R⁷;
R¹ is hydrogen;
R² is C₁-C₈alkyl or C₁-C₈alkyl substituted by halogen, C₃-C₁₀cycloalkyl or C₃-C₁₀cycloalkyl substituted by one or two methyl groups, phenyl-C₁-C₄alkylene- or phenyl-C₁-C₄alkylene- wherein the phenyl moiety is substituted by one to five R¹⁰, pyridyl-C₁-C₄alkylene- or pyridyl-C₁-C₄alkylene- wherein the pyridyl moiety is substituted by one to four R¹⁰, oxetanyl, thietanyl, oxo-thietanyl, dioxo-thietanyl;
R³ is trifluoromethyl;
R⁴ is 3,5-dichloro-phenyl;
R⁵ is methyl;
R^{6a} is hydrogen;
R^{6b} is methyl or phenyl;
R⁷ is hydroxy;
R¹⁰ is bromo, chloro, fluoro, cyano or methyl.

11. A compound of formula (IIa) 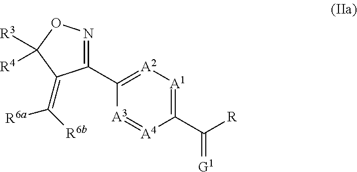
A¹, A², A³, A⁴, G¹, R³, R⁴, R^{6a}, and R^{6b} are as defined in claim 1, and R is hydroxy, C₁-C₆alkoxy or halogen; or a salt or N-oxide thereof; or
a compound of formula (IIa′) 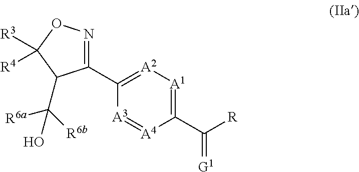
wherein A¹, A², A³, A⁴, G¹, R³, R⁴, R^{6a} and R^{6b} are as defined in claim 1, and R is as defined for a compound of formula (IIa); or a salt or N-oxide thereof, or
a compound of formula (IIb) 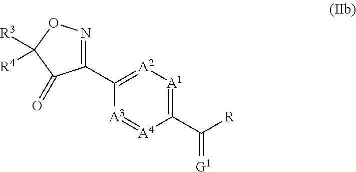
wherein A¹, A², A³, A⁴, G¹, R³ and R⁴ are as defined in claim 1, and R is hydroxy, C₁-C₆alkoxy or halogen; or a salt or N-oxide thereof; or
a compound of formula (IIc) 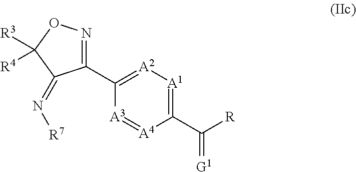
wherein A¹, A², A³, A⁴, G¹, R³, R⁴ and R⁷ are as defined in claim 1, and R is hydroxy, C₁-C₆alkoxy or halogen; or a salt or N-oxide thereof.

12. A method of combating and/or controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I) as defined in claim 1.

13. An insecticidal, acaricidal, nematicidal or molluscicidal composition comprising an insecticidally, acaricidally, nematicidally or molluscicidally effective amount of a compound of formula (I) as defined in claim 1.

14. An insecticidal, acaricidal, nematicidal or molluscicidal composition according to claim 13 comprising an additional compound having biological activity.
